(19) United States
(12) Patent Application Publication
(10) Pub. No.: US 2024/0324870 A1
(43) Pub. Date: Oct. 3, 2024

(54) MEDICAL INSTRUMENT GUIDANCE SYSTEMS, INCLUDING GUIDANCE SYSTEMS FOR PERCUTANEOUS NEPHROLITHOTOMY PROCEDURES, AND ASSOCIATED DEVICES AND METHODS

(71) Applicant: INTUITIVE SURGICAL OPERATIONS, INC., Sunnyvale, CA (US)

(72) Inventors: Serena H. Wong, Los Altos, CA (US); Zachary Moller, San Francisco, CA (US); Gavin Jensen, Saratoga Springs, UT (US); Tabish Mustufa, Sunnyvale, CA (US)

(21) Appl. No.: 18/699,388

(22) PCT Filed: Oct. 6, 2022
(86) PCT No.: PCT/US2022/077700

§ 371 Date: Apr. 8, 2024

Related U.S. Application Data
(60) Provisional application No. 63/253,915, filed on Oct. 8, 2021.

(51) Int. Cl. A61B 1/307; A61B 34/10

(52) U.S. Cl. CPC A61B 1/307; A61B 34/10; A61B 2034/105; A61B 2034/107

(57) ABSTRACT

Medical instrument guidance systems and associated devices and methods are disclosed herein. In some embodiments, a method for providing guidance for percutaneous access to a target within an anatomic structure, includes receiving point cloud data from a sensor system coupled to an internal instrument as the internal instrument is moved within the anatomic structure; generating a 3D model of the anatomic structure based at least in part on the point cloud data; and receiving information for identifying a substructure within the 3D anatomic model. The substructure can provide access to the target. The method can further include determining an entry to the substructure; determining an approach path through the entry; and providing a graphical representation of the approach path.

### CROSS-REFERENCED APPLICATIONS

This application claims priority to and benefit of U.S. Provisional Application No. 63/253,915, filed Oct. 8, 2021 and entitled “Medical Instrument Guidance Systems, Including Guidance Systems for Percutaneous Nephrolithotomy Procedures, and Associated Devices and Methods,” which is incorporated by reference herein in its entirety.

### TECHNICAL FIELD

The present disclosure is directed to systems and associated devices and methods for providing guidance for medical procedures. For example, several embodiments of the present technology are directed to guidance systems for percutaneous nephrolithotomy (PCNL) procedures.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Some minimally invasive medical tools may be teleoperated or otherwise computer-assisted or delivered by a teleoperated, robotic, or otherwise computer-assisted system. Various features may improve the effectiveness of minimally invasive medical tools and techniques.

### SUMMARY

Embodiments of the present technology are best summarized by the claims that follow the description.

In some embodiments, a method for providing guidance for percutaneous access to a target within an anatomic structure comprises receiving point cloud data from a sensor system coupled to an internal instrument as the internal instrument is moved within the anatomic structure. The method can further include generating a 3D model of the anatomic structure. The 3D model can be based on the point cloud data. The method can also include receiving information for identifying a substructure within the 3D anatomic model. The substructure can provide access to the target. The method can further include determining an entry to the substructure and determining an approach path through the entry. The method can also include providing a graphical representation of the approach path to the target based at least in part on geometry of the substructure.

In these and other embodiments, a system for providing guidance for percutaneous access to a target within an anatomic structure comprises an instrument including a sensor system. The sensor system can include a first sensor for capturing point cloud data and a second sensor for capturing imaging data. The system can further include a processor operably coupled to the sensor system, and a memory operably coupled to the processor. The memory can store instructions that, when executed by the processor, cause the system to perform various operations. The operations can include generating a 3D model of the anatomic structure based on the point cloud data. The operations can further include receiving the localization data and the imaging data to identify the target within the anatomic structure and a substructure within the anatomic structure. The substructure can provide access to the target. The operations can also include determining an approach path to the target through a distal entry of the sub-structure. The system can further include a display for providing the 3D model of the anatomic structure and a graphical representation of the approach path to the target within the 3D model.

In these and further embodiments, a non-transitory, computer-readable medium is provided. The non-transitory, computer-readable instructions stores instructions thereon that, when executed by one or more processors of a computing system, cause the computing system to perform the method of any of the embodiments described herein.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a flow diagram illustrating a method for performing a medical procedure in accordance with various embodiments of the present technology.

FIG. 2 is a flow diagram illustrating a method for generating a 3D model of an anatomic structure in accordance with various embodiments of the present technology.

FIG. 3 is a partially schematic illustration of an anatomic structure and an elongate flexible device within the anatomic structure, in accordance with various embodiments of the present technology.

FIG. 4 illustrates a representative example of point cloud data generated in accordance with various embodiments of the present technology.

FIG. 5 illustrates a representative examples of a 3D anatomic model generated in accordance with various embodiments of the present technology.

FIGS. 6-10 illustrate various approach paths to a target via anatomic substructures, in accordance with various embodiments of the present technology.

FIGS. 11A-12 illustrate various examples of graphical user interfaces for providing guidance for deploying an access tool, in accordance with various embodiments of the present technology.

FIG. 13 is a simplified diagram of a teleoperated medical system configured in accordance with various embodiments of the present technology.

FIG. 14A is a simplified diagram of a medical instrument system configured in accordance with various embodiments of the present technology.

FIG. 14B is a simplified diagram of a medical instrument system configured in accordance with various embodiments of the present technology.

### DETAILED DESCRIPTION

The present disclosure is directed to minimally invasive devices, systems, and methods for providing guidance for medical procedures. In some embodiments, a medical procedure includes introducing an elongate flexible device (e.g., a flexible catheter, an endoluminal instrument, a ureteroscope) into an anatomic structure (e.g., a kidney) of a patient. The elongate flexible device can include at least one sensor configured to locate at least one target (e.g., a kidney stone) in the anatomic structure. Once the target location has been identified, an access tool (e.g., a needle) can be used to create an access path to the target. The access path can be a percutaneous access path for introducing a medical instrument from a location external to the anatomic structure to a location of the target internal the anatomic structure. In some embodiments, for example, the medical instrument can be a tool (e.g., a suction tube, nephroscope, or lithotripter) for breaking up a kidney stone via a PCNL procedure.

In such medical procedures, it may be challenging for the operator to (a) identify a percutaneous path to the target location that avoids sensitive organs and/or other anatomic structures, and/or (b) navigate the access tool along the identified path. For example, in a PCNL procedure, the operator may need to create a percutaneous access path to a kidney stone (i) without intersecting ribs and/or the sides or walls of the kidney and/or (ii) without puncturing the liver, intestines (e.g., bowels, colon, etc.), lungs, and/or nearby blood vessels. Continuing with this example, once an access path has been identified, the operator may require guidance to navigate the access tool to the kidney stone. Conventional techniques, however, may not provide sufficient guidance for positioning the access tool. For example, preoperative imaging and/or modeling may be of limited value because the position of the kidney stone, kidney, and/or other organs may shift, e.g., due to differences in the patient's body position during preoperative imaging versus the actual PCNL procedure. Additionally, the kidney and/or surrounding organs can be soft, deformable structures that may change in shape and/or size after preoperative imaging. Additionally, kidney stones may not be visible in certain imaging modalities (e.g., fluoroscopy, computed tomography (CT)). Thus, conventional procedures may rely upon highly trained specialists to perform the initial puncture with the access tool and/or may frequently require multiple attempts to create an access path that is sufficiently on target.

To overcome these and other challenges, the systems and associated methods described herein can be configured to guide an operator in creating an access path to an anatomic target while avoiding nearby sensitive tissue structures. In some embodiments, for example, the system generates an intraoperative 3D model of an anatomic structure (e.g., a kidney) and a representation of a target (e.g., a kidney stone) within the anatomic structure using an elongate flexible device (e.g., a catheter) deployed within the anatomic structure. The elongate flexible device can include an imaging system (e.g., an endoscopic camera) and a sensor system (e.g., a shape sensor) configured to obtain data (e.g., localization data, point cloud data, image data) used to determine the 3D shape of the anatomic structure and identify the location of the target. Using the 3D model, the system identifies one or more access paths for an access tool (e.g., a needle) to reach the target along an approach path from a location external the anatomic structure, through an identified anatomic substructure, and to a location of the target. For example, the system determines an access path that approaches a kidney stone through a distal opening of a calyx, reducing or minimizing contact with kidney walls (e.g., walls of calyces) and reducing or minimizing excessive puncturing of the kidney wall if multiple approaches must be taken. Also, because blood vessels typically run alongside the walls of a kidney, approaching a kidney stone through a distal opening of a calyx can avoid sides or walls of the calyx or the kidney, thereby reducing or minimizing the risk of puncturing the blood vessels or other sensitive anatomic structures. In some cases, multiple paths through different anatomic substructures to the target may be identified and/or available. Accordingly, the 3D model can also include locations of sensitive anatomic structures to be avoided, and the system may identify an optimal path based at least in part on avoiding such sensitive anatomic structures. Additionally, or alternatively, the system can rely on the pointing direction of the elongate flexible instrument when directed towards the anatomic substructure to determine the approach path into the anatomic substructure. In some embodiments, the system can output a graphical user interface that provides (e.g., accurate and/or real-time) guidance for positioning the access tool (e.g., acceptable insertion locations, acceptable range of insertion angles, navigation rings or icons) to create the access path.

Accordingly, the present technology is expected to simplify PCNL and other percutaneous medical procedures (a) by assisting an operator to identify appropriate approach paths to a target location within an anatomic structure that avoid puncturing the wall of an organ and avoid sensitive organs and other structures and (b) by assisting the operator to navigate an access tool along the approach path to create an access path. In turn, the present technology is expected to reduce the likelihood of inadvertent injury to organs and blood vessels and surrounding tissues while creating an access path during the procedure that can improve efficacy of such procedures by enabling more optimal positioning and reach of the associated tools. In addition, the present technology is expected to reduce the number of attempts to create an access path that is sufficiently on target. Thus, the present technology is expected to reduce the time required to conduct such procedures. Furthermore, the present technology is expected to reduce reliance on highly trained professionals to perform the initial puncture with and/or navigation of an access tool to a target location.

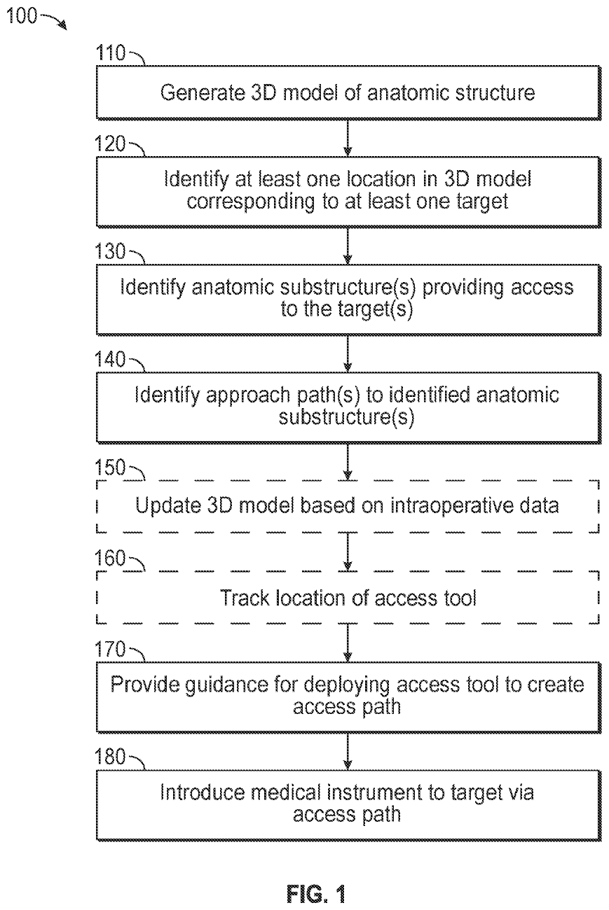

Specific details of several embodiments of the present technology are described herein with reference to FIGS. 1-14B. Although many of the embodiments are described below in the context of navigating and performing medical procedures within a kidney and/or urinary tract of a patient, other applications and other embodiments in addition to those described herein are within the scope of the present technology. For example, unless otherwise specified or made clear from context, the devices, systems, and methods of the present technology can be used for navigating and performing medical procedures on, in, or adjacent other patient anatomy, such as the lungs, heart, uterus, bladder, prostate, and/or other components of the urinary system, circulatory system, and/or gastrointestinal (GI) system of a patient.

It should be noted that other embodiments in addition to those disclosed herein are within the scope of the present technology. For example, although certain embodiments herein are discussed with reference to instruments for accessing and/or breaking up kidney stones, this is not intended to be limiting, and the present technology can also be applied to other types of medical instruments, such as instruments used for diagnosis, treatment, or other medical procedures. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

As used herein, the term “operator” shall be understood to include any type of personnel who may be performing or assisting a procedure and, thus, is inclusive of a physician, a surgeon, a doctor, a nurse, a medical technician, a clinician, other personnel or user of the technology disclosed herein, and any combination thereof. As used herein, the term “patient” should be considered to include human and/or non-human (e.g., animal) patients upon which a medical procedure is being performed.

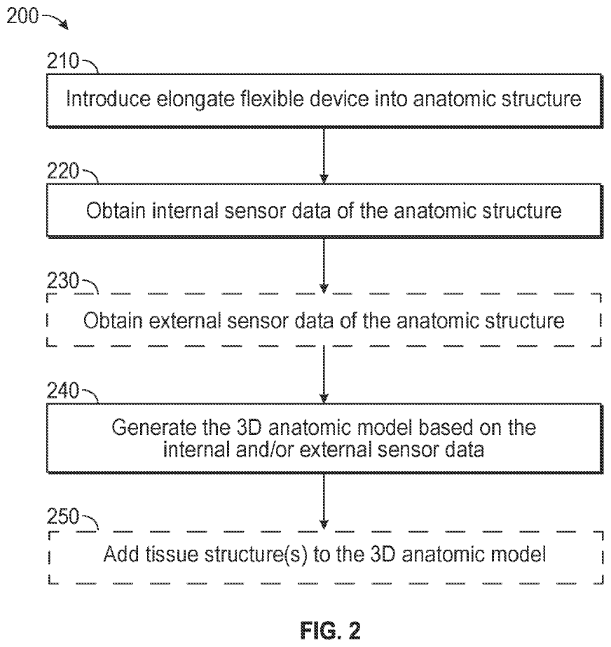

FIG. 1 is a flow diagram illustrating a method 100 for performing a medical procedure in accordance with various embodiments of the present technology. The method 100 is illustrated as a set of steps or processes 110-180. All or a subset of the steps of the method 100 can by implemented by any suitable computing system or device, such as a control system of a medical instrument system or device (e.g., including various components or devices of a robotically-controlled or teleoperated surgical system), a workstation, a portable computing system (e.g., a laptop computer), any/or a combination thereof. In some embodiments, for example, the computing system for implementing the method 100 includes one or more processors operably coupled to a memory storing instructions that, when executed, cause the computing system to perform operations in accordance with the steps 110-180. Additionally or alternatively, all or a subset of the steps 110-180 of the method 100 can be executed at least in part by an operator (e.g., a physician, a user, etc.) of the computing system, and/or by a robotically-controlled surgical system via user inputs from the operator through a user input device or automatically through using closed loop control and/or pre-programmed instructions through a processor of the system. The method 100 is illustrated in the following description by cross-referencing various aspects of FIGS. 2-14B.

The method 100 begins at step 110 with generating a three-dimensional (“3D”) model of the anatomic structure (also referred to herein as a “3D anatomic model”). The 3D anatomic model can be any suitable 3D representation of the passageways, spaces, and/or other features of the anatomic structure, such as a surface model (e.g., a mesh model or other representation of anatomic surfaces, a skeletal model (e.g., a model representing passageways and/or connectivity), or a parametric model (e.g., a model fitting common parameters). As described in greater detail below, the 3D anatomic model can include a representation of at least one target, which can be a tissue, object, or any other suitable site to be accessed and/or treated during the medical procedure. For example, in embodiments where the anatomic structure is a kidney, the 3D anatomic model can include representations of major calyces, minor calyces, a renal pelvis, and/or a ureter, and the target can be a kidney stone within the kidney. In other embodiments, however, the 3D anatomic model can include representations of other types of anatomic structures and/or targets.

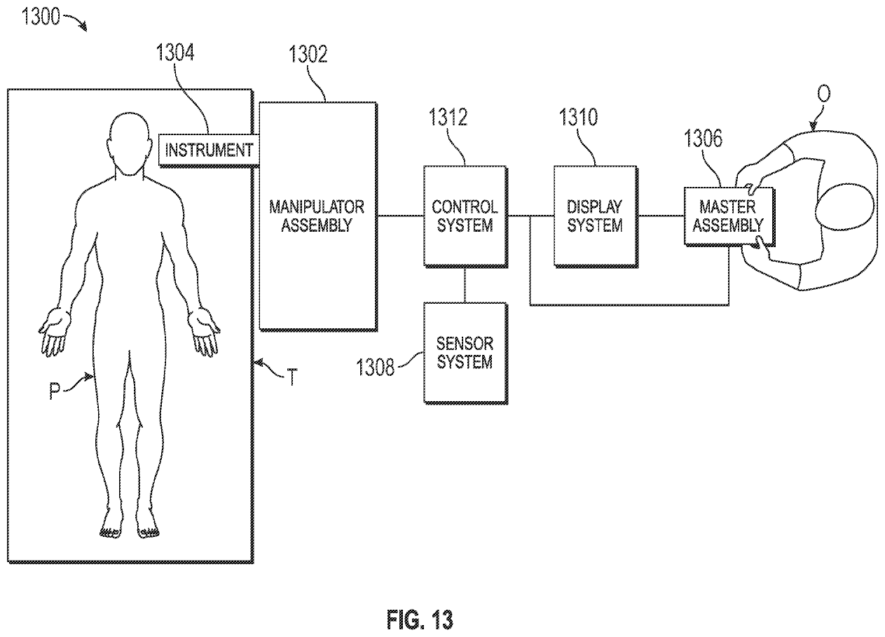

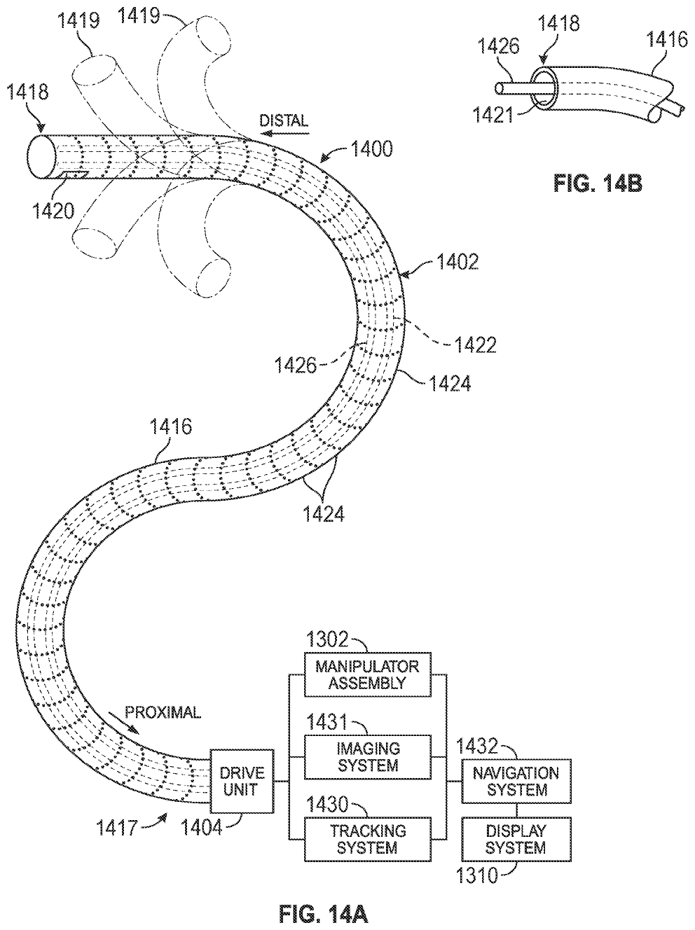

FIG. 2 is a flow diagram illustrating a method 200 for generating a 3D anatomic model that can be performed at step 110 of the method 100 (FIG. 1) in accordance with various embodiments of the present technology. The method 200 begins at step 210 with introducing an elongate flexible device into an anatomic structure of a patient. The elongate flexible device can be a flexible catheter, an endoluminal instrument, a ureteroscope, or another similar tool suitable for introduction into the anatomic structure via minimally invasive techniques (e.g., via an endoluminal access route). Positioning and/or navigation of the elongate flexible device may be performed manually, the elongate flexible device may be robotically controlled by an operator via an input device, and/or the elongate flexible device may be robotically controlled automatically using a pre-programmed set of instructions from a robotic system. Additional details of elongate flexible devices and robotic medical systems suitable for use with the method 100 are provided below with reference to FIGS. 13-14B.

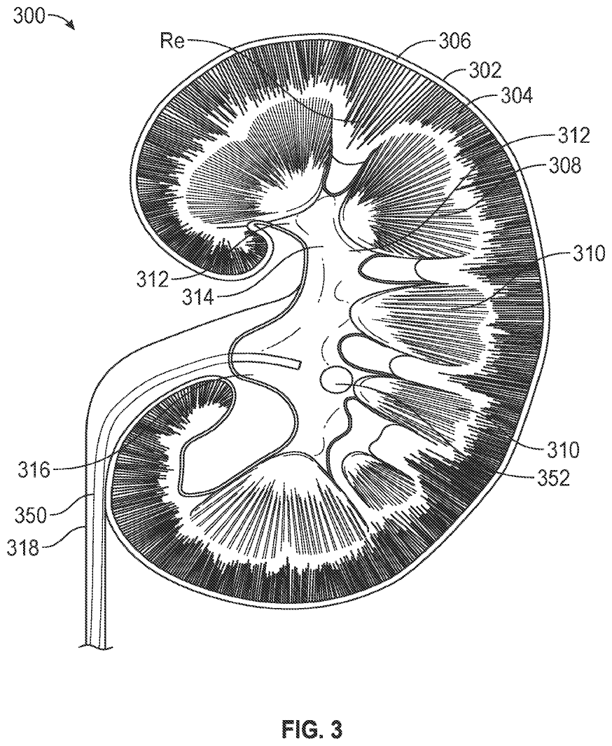

FIG. 3, for example, is a partially schematic illustration of an anatomic structure 300 and an elongate flexible device 350 within the anatomic structure 300 in accordance with various embodiments of the present technology. In the illustrated embodiment, the anatomic structure 300 is a patient's kidney 302. The kidney 302 includes a renal capsule 304, a renal cortex 306, and a renal medulla 308. The renal medulla 308 includes a plurality of renal pyramids 310 containing the nephron structures responsible for urine production. The urine is collected by a series of chambers or lumens known as calyces (e.g., minor calyces 312 and major calyces 314). The minor calyces 312 are adjacent to the renal pyramids 310 and converge to form major calyces 314. The major calyces 314 empty into the renal pelvis 316 and ureter 318.

The elongate flexible device 350 can be an endoluminal instrument such as a catheter, a ureteroscope, a guide wire, a stylet, or another similar instrument suitable for introduction into the kidney 302 via the patient's urinary tract (e.g., the ureter 318). The elongate flexible device 350 can navigate and/or articulate within the interior spaces of the kidney 302 to reach a target 352 (e.g., a kidney stone). The target 352 may be located near or within the minor calyces 312, major calyces 314, renal pelvis 316, or ureter 318.

Referring again to FIG. 2, the 3D anatomic model can be generated partially or entirely from intraoperative data obtained during the medical procedure (e.g., while the elongate flexible device is positioned within the anatomic structure). The intraoperative data can include location data (e.g., point cloud data) generated continuously by a localization sensor coupled to the elongate device as the elongate flexible device moves within the anatomic structure. The process of navigating the elongate flexible device within the anatomic structure while obtaining and saving location data generated by the localization sensor may also be referred to herein as “surveying” the anatomic structure, and the location data generated during the surveying process may be referred to herein as “survey location data.” As previously described, location data and/or other intraoperative data may provide a more accurate representation of the current state of the patient anatomy and/or target, compared to preoperative data (e.g., preoperative CT, X-ray, MRI images and/or models) which may be captured a long period of time before performing the medical procedure and/or while a patient is positioned differently than during the medical procedure.

In particular, the method 200 of FIG. 2 can continue at step 220 with obtaining internal sensor data of an anatomic structure (e.g., an anatomic cavity, such as the interior spaces of a kidney or other organ). The internal sensor data can include, for example, sensor data generated by a sensor system carried by the elongate flexible device. For example, the sensor system can be, or can include, at least one localization sensor configured to generate survey location data as the elongate flexible device surveys the anatomy by driving to various locations within the anatomic structure. The survey location data can be saved to create a cloud of points forming a general shape of the anatomic structure. Any suitable localization sensor can be used, such as a shape sensor, EM sensor, positional sensor, pose sensor, or a combination thereof. The localization sensor may be integrated within the elongate flexible device. For example, the localization sensor may be integrated within a catheter or ureteroscope, or integrated within a stylet or guide wire insertable within the catheter or ureteroscope.

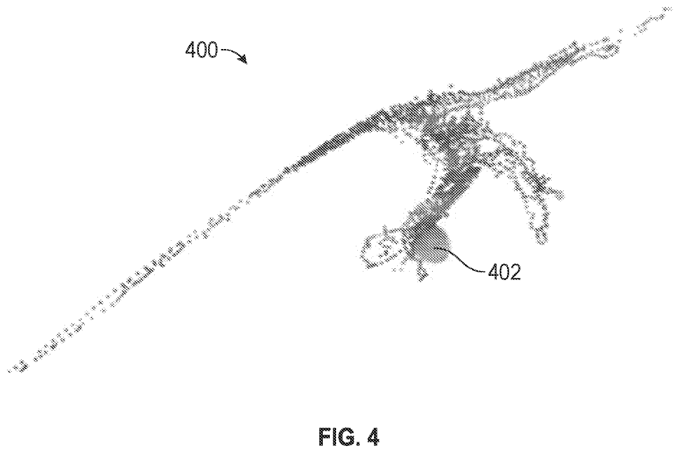

FIG. 4 illustrates a representative example of a point cloud data set 400 generated in accordance with embodiments of the present technology. The point cloud data set 400 can be generated, for example, by navigating the elongate flexible device to different locations within the anatomic structure, and can provide a 3D representation of the interior spaces and/or passageways of the anatomic structure. In the illustrated embodiment, for example, the point cloud data set 400 depicts the 3D shape of a ureter, renal pelvis, major calyces, and minor calyces of a patient's kidney. The point cloud data set 400 also includes a set of data points corresponding to the location of a target 402 (e.g., a kidney stone) within the anatomic structure. Optionally, the point cloud data set 400 can include data of additional locations within or near the anatomic structure to provide an accurate representation of the relative shape of the anatomy and the location of the target. The point cloud data set 400 can be used to generate a 3D anatomic model of the kidney and kidney stone, as disclosed herein.

Referring again to step 220 of FIG. 2, in some embodiments, the internal sensor data includes other types of data in addition to location data. For example, the internal sensor data can include image data generated by an imaging device within the anatomic structures (e.g., carried by the elongate flexible device). The image data can include, for example, still or video images, ultrasound data, thermal image data, and the like. In some embodiments, each image captured by the imaging device is associated with location data generated by the localization sensor, such that the location of an object within the anatomic structure can be determined based on images of the object and the location data associated with the images.

At step 230, the method 200 can optionally include obtaining external sensor data of the anatomic structure. The external sensor data can include any data generated by a sensor system external to the patient's body, such as external imaging data generated by an external imaging system. The external image data can include any of the following: CT data, magnetic resonance imaging (MRI) data, fluoroscopy data, thermography data, ultrasound data, optical coherence tomography (OCT) data, thermal image data, impedance data, laser image data, nanotube X-ray image data, and/or other suitable data representing the patient anatomy. The image data can correspond to two-dimensional (2D), 3D, or four-dimensional (e.g., time-based or velocity-based information) images. In some embodiments, for example, the image data includes 2D images from multiple perspectives that can be combined into pseudo-3D images. The external sensor data can include preoperative data and/or intraoperative data.

At step 240, the method 200 continues with generating the 3D anatomic model based on the internal and/or external sensor data. For example, the 3D anatomic model can be generated from the survey location data (e.g., point cloud data) using techniques for producing a surface or mesh model from a plurality of 3D data points, such as a surface reconstruction algorithm. In such embodiments, because the sensor system used to generate the point cloud data is carried by the elongate flexible device, the resulting 3D anatomic model may already be in the same reference frame as the elongate flexible device, such that no additional registration step is needed. As another example, a 3D representation can be generated from preoperative image data (e.g., using image segmentation processes), and subsequently combined with the point cloud data to produce the 3D anatomic model. In such embodiments, the method 200 can further include determining a registration between the image data and the point cloud data (e.g., using a registration algorithm, such as a point-based iterative closest point (ICP) technique, as described in U.S. Provisional Pat. App. Nos. 62/205,440 and No. 62/205,433, which are both incorporated by reference herein in their entireties).

Optionally, the 3D anatomic model can be generated from both intraoperative data (e.g., internal sensor data, such as location data) and preoperative data (e.g., external image data obtained before the elongate flexible device is introduced into the patient's body). In such embodiments, the intraoperative data can be used to update the preoperative data to ensure that the resulting model accurately represents the current state of patient anatomy. For example, a preoperative anatomic model can be generated from image data (e.g., CT data) and/or other patient data obtained before the medical procedure (e.g., using image segmentation processes known to those of skill in the art). Subsequently, the preoperative anatomic model can be registered to the intraoperative data (e.g., point cloud data) to place them both in the same reference frame. The registration process can include navigating and/or touching the elongate flexible device to locations of the patient anatomy (e.g., within the anatomic structure) corresponding to known points in the preoperative anatomic model. Alternatively, or in combination, the intraoperative data can be registered to the preoperative anatomic model using a registration algorithm (e.g., a point-based ICP technique). Once registered, the intraoperative data can be used to modify the preoperative anatomic model (e.g., by filling in missing portions, resolving errors or ambiguities, etc.). If there are portions of the preoperative model that do not match the intraoperative data, the intraoperative data can be assumed to be more accurate and can be used to replace those portions of the preoperative model. Additionally, or alternatively, portions and/or features (e.g., overall shape) of the 3D model can be generated and/or based at least in part on well-known, average patient data or anatomy.

At step 250, the method 200 can optionally include adding one or more tissue structures to the 3D anatomic model. In some embodiments, the tissue structures can include sensitive tissue structures, such as any tissue, organ, or other site to be avoided during the medical procedure (e.g., due to risk of injury, side effects, and/or other complications). The sensitive tissue structures can be located nearby but outside of the anatomic structure to be treated. For example, sensitive tissue structures in the context of a kidney-related procedure (e.g., a PCNL procedure) can include the patient's liver, intestines, lungs, and/or blood vessels. In some embodiments, step 250 includes generating one or more model components representing the geometry and/or locations of the skin or sensitive tissue structures, and adding the model components to the 3D anatomic model. Alternatively, or in combination, step 250 can include marking or otherwise identifying existing components or locations within the 3D anatomic model as corresponding to the locations of the sensitive tissue structures.

In some embodiments, in order to add the sensitive tissue structures to the appropriate locations in the 3D anatomic model, step 250 of the method 200 further includes determining the geometry and/or locations of the sensitive tissue structures relative to the anatomic structure. For example, the geometry and/or locations of the sensitive tissue structures can be estimated based on general anatomic information (e.g., the expected geometry and/or locations for a standard patient) and/or characteristics of the particular patient (e.g., age, sex, height, weight). As another example, the geometry and/or locations of the sensitive tissue structures can be determined based on preoperative or intraoperative data (e.g., CT images). In a further example, the locations of the sensitive tissue structures can be estimated based on known spatial relationships (e.g., knowledge of how the elongate flexible device is positioned relative to the anatomic structure, how the insertion stage for the elongate flexible device is positioned relative to the surgical table, how the patient's body is positioned on the table, and/or where the sensitive tissue structures are generally located in the patient's body). In yet another example, the locations of the sensitive tissue structures can be estimated by obtaining location data of known anatomic reference points with the elongate flexible device. For instance, a localization sensor can track the location of the elongate flexible device as the elongate flexible device is touched to one or more external and/or internal anatomic reference points (e.g., the ribs), and the tracked location can be used to register the anatomic reference points to the 3D anatomic model. The location of the sensitive tissue structures can then be estimated based on known spatial relationships between the sensitive tissue structures and the anatomic reference points.

In still other embodiments, the locations of the sensitive tissue structures can be estimated based on user input from the operator, a physician, or other healthcare professional. For example, a physician could estimate the locations of sensitive tissue structures in the patient, e.g., by manually palpating the patient. The physician (or another operator) could mark these locations and/or other anatomy (e.g., the patient's ribs) by touching the elongate flexible device or another sensor (e.g., a shape sensor, an EM sensor, a tracked needle, a tracked stylet, etc.) to the corresponding locations on the patient's external and/or internal anatomy. The marked locations can be used to define a space or region that should be avoided during the procedure. For example, the physician (or another operator) can trace a stylet or another tool along the patient's skin or ribs to identify or define a zone or region that should be avoided while creating a percutaneous puncture or an access path to a target. In other embodiments, sensors (e.g., location sensors integrated into a patient patch or other structure) may be coupled to patient anatomy at locations of sensitive tissue. In other embodiments, sensors (e.g., location sensors integrated into a patient patch or other structure) may be coupled to patient anatomy at locations of sensitive tissue.

In some embodiments, adding one or more tissue structures to the 3D anatomic model can include adding a rendering of the patient's skin surrounding the anatomic structure using, for example, external imaging of the patient or one or more external sensors or markers. In embodiments in which external imaging is used to add a rendering of the patient's skin surrounding the anatomic structure, the external images can be registered to point cloud data captured using the elongate flexible device internal the anatomic structure. For example, the external images can be registered to the point cloud data by touching an external sensor (e.g., a shape sensor, an EM sensor, a tracked needle, a tracked stylet, etc.) to portions of the patient's anatomy before, during, and/or after collecting data points for the point cloud of the anatomic structure. Additionally, or alternatively, an external sensor (e.g., a stylet, a needle, etc.) can be traced over the surface of the patient's skin and/or over other critical features (e.g., the patient's ribs) to add data points to the point cloud data of the 3D model and to register the external sensor to the point cloud data. Such added data points can indicate valid percutaneous entry points and/or off-limit areas on the patient's skin for percutaneous entry points. Such added data points can also provide information regarding a distance between patient's skin and a tip of the elongate flexible device positioned internal the anatomic structure.

In some embodiments, the geometry and/or locations of the sensitive tissue structures and/or the patient's skin determined in step 250 can be initial estimates, and the 3D anatomic model can subsequently be further updated to refine these estimates, if appropriate. The process for updating the 3D anatomic model is described further below with reference to step 150 of FIG. 1.

Referring again to FIG. 1, the method 100 continues at step 120 with identifying at least one location in the 3D anatomic model corresponding to at least one target within the anatomic structure. As previously discussed, the target can be an object (e.g., a kidney stone), a tissue to be treated (e.g., biopsied, ablated, etc.), or any other suitable site within the anatomic structure. In some embodiments, the target location can be identified, for example, based on internal sensor data generated by a sensor system carried by the elongate flexible device. For example, the sensor system can include an imaging device (e.g., a camera, ultrasound, OCT, etc.) configured to obtain image data of the target. In such embodiments, the elongate flexible device can be navigated within the anatomic structure until the target is within the field of view of the imaging device and is at least partially visible within the image data. The process of imaging and identifying the target can be performed automatically, can be performed based at least in part on user input, or suitable combinations thereof. For example, an operator can view the image data (e.g., via a graphical user interface shown on a monitor), and can provide commands via an input device (e.g., touchscreen, mouse, keyboard, joystick, trackball, button, etc.) to indicate the presence of the target in the image data (e.g., by clicking, selecting, marking, etc.). As another example, the operator can drive the elongate flexible device until the target is at a particular location in the image data (e.g., aligned with a visual guide such as a set of crosshairs, centered in the image data, etc.). In yet another example, the method 100 can include analyzing the image data using computer vision and/or machine learning techniques to automatically or semi-automatically identify the target.

Once the target is visible in the image data, step 120 can further include obtaining target location data using a localization sensor (e.g., a shape sensor or EM sensor), and determining the location of the target with respect to the 3D anatomic model based on the target location data and the image data. The target location data obtained in step 120 can be different from the survey location data used to generate in the 3D anatomic model in step 110, or can include some or all of the same data points as the target location data. Similarly, the localization sensor can be the same sensor used to obtain the survey location data in step 110, or can be a different sensor. The target location data can indicate the pose of the elongate flexible device while the target is within the field of view of the imaging device. Thus, the target location data can be used to calculate the spatial relationship between the target and the elongate flexible device, which in turn can be used to determine the location of the target in the 3D anatomic model. In embodiments where two different localization sensors are used to generate the survey location data and the target location sensor data, if the relative positions of the two localization sensors are known (e.g., the sensors are both coupled to the elongate flexible device), the target location data can be registered to the survey location data so a representation of the target can be positioned appropriately within the 3D anatomic model.

In some embodiments, step 120 of the method 100 also includes determining the distance between the target and the elongate flexible device (or a portion thereof, such as the distal end portion). The distance can be determined in many different ways. For example, the distance can be measured using a proximity sensor (e.g., an optical sensor, time-of-flight sensor, etc.) carried by the elongate flexible device. Alternatively, or in combination, the distance can be determined based on the known or estimated geometry (e.g., diameter, height, width) of the target. In such embodiments, the target geometry can be determined or estimated based on image data (e.g., preoperative images) or any other suitable data. Subsequently, the target geometry can be compared to the geometry of the target in the image data to determine the distance between the target and the imaging device (and thus, the elongate flexible device carrying the imaging device). Based on the determined distance, a representation of the target can be added to 3D anatomic model at the appropriate location.

Alternatively, or in combination, step 120 of the method 100 can include using force, pressure, and/or contact sensor(s) carried by the elongate flexible device to detect the target. This approach can be used in situations where the target has different characteristics or properties than the surrounding tissue, such as a different hardness and/or stiffness. In such embodiments, the elongate flexible device can be navigated within the anatomic structure until the force and/or contact sensor detects that the elongate flexible device is in contact with the target. The location of the elongate flexible device (or a portion thereof, such as the distal end portion) at the time of contact can be used as the location of the target.

In some embodiments, identifying the at least one location can include adding at least one representation of at least one target to the 3D anatomic model. For example, step 120 of the method 100 can include generating a model component (e.g., a representation) representing the target and adding that model component to the 3D anatomic model. Alternatively, or in combination, step 120 can include marking an existing model component and/or location in the 3D anatomic model that corresponds to the location of the target in the anatomic structure.

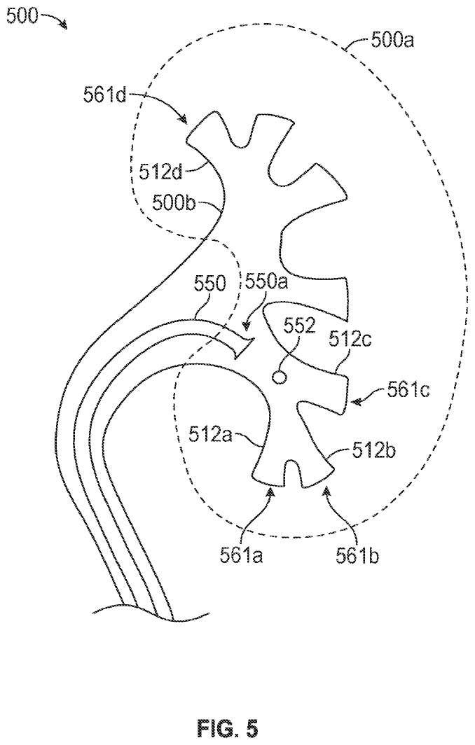

FIG. 5 illustrates a representative example of a 3D anatomic model 500 generated in accordance with various embodiments of the present technology. As shown, the 3D anatomic model 500 includes a representation 500a of the overall shape of an anatomic structure. In FIG. 5, the anatomic structure is a kidney, and the overall shape of the kidney can be estimated and/or based on external imaging and/or well-known patient data. The 3D anatomic model 500 also includes a representation 500b of anatomic substructures (e.g., kidney calyces, a renal pelvis, and a ureter). Thus, the representation 500b of the 3D model 500 includes representations 512 of kidney calyces (some of which are identified individually as representations 512a-512d (“calyces 512a-512d”) in FIG. 5) generated, for example, based on point cloud data captured by the elongate flexible device positioned within the kidney and/or on external imaging. The 3D anatomic model 500 further includes a representation 550 of the elongate flexible device and a representation 552 of a target (e.g., a kidney stone) within the kidney. The representation 550 of the elongate flexible device can be shown with a position, shape, and/or orientation within the 3D model that corresponds to the position, shape, and/or orientation of the elongate flexible device within the kidney. The position, shape, and/or orientation of the elongate flexible device can be determined using one or more sensors (e.g., a shape sensor, one or more position sensors, etc.) positioned at the tip and/or at other locations along the elongate flexible device. Alternatively, the representation 550 of the elongate flexible device can be shown within the 3D model with a position, shape, and/or orientation that represents an estimate of the position, shape, and/or orientation of the (e.g., tip portion of the) elongate flexible device within the kidney. The estimate can be based, for example, on one or more sensors positioned on the elongate flexible device. Similarly, the representation 552 of the target is positioned within the 3D anatomic model 500 at a location corresponding to the location of the target within the kidney.

Returning to FIG. 1, at step 130, the method 100 continues with identifying one or more anatomic substructures that provide access to the target location(s). As discussed above, the anatomic structure can be a patient's kidney, and anatomic substructures can include kidney calyces. In such embodiments, optimal approach paths for an access tool during a PCNL procedure can include paths that enter the kidney via distal openings of calyces that provide access to the target location(s). For example, an optimal approach path may be a path that enters a distal opening of a calyx in which a kidney stone is positioned. Additionally, or alternatively, an optimal approach path may be a path that enters a distal opening of a calyx that provides access to a kidney stone (but may or may not be a calyx in which the kidney stone is positioned). In these and still other embodiments, an optimal approach path may be a path that enters a distal opening of a calyx with an access tool oriented generally parallel with the calyx. As discussed above, entering a kidney through a distal opening of a calyx can avoid pressing on or puncturing walls of the kidney and/or puncturing patient blood vessels that extend along the walls of the kidney. In addition, entering a distal opening of a calyx with an access tool oriented generally parallel with the calyx can avoid puncturing walls of the calyx and/or otherwise (e.g., unnecessarily) perforating the urinary system of the patient.

In some embodiments, identifying one or more anatomic substructures that provide access to a target location can include identifying one or more anatomic substructures based at least in part on a position of the target within the 3D anatomic model relative to the location of anatomic substructures in the 3D anatomic model. For example, referring again to FIG. 5, the representation 552 of the target is positioned proximate the calyces 512a-512c, and each of the calyces 512a-512c provide access to the location of the target via distal openings 561a-561c, respectively, of the calyces 512a-512c in the 3D anatomic model. Thus, all or a subset of the calyces 512a-512c can be identified at step 130 of the method 100 as anatomic substructures that provide access to the target 552. As another example, the calyx 512d may also be identified at step 130 as an anatomic substructure that provides access to the target 552 based at least in part on the fact that the calyx 512d provides direct (e.g., linear) access to the target 552 via a distal opening 561d of the calyx 512d.

In these and other embodiments, identifying one or more anatomic substructures that provide access to a target location can include identifying one or more anatomic substructures based at least in part on the elongate flexible device positioned within the anatomic structure. For example, any of the calyces 512a-512c in FIG. 5 can be identified at step 130 of the method 100 based at least in part on their proximity to a tip portion 550a of the elongate flexible device 550. As another example, anatomic substructures can be identified at step 130 of the method 100 based at least in part on a pointing direction of the elongate flexible device 550 and/or on the tip portion 550a of the elongate flexible device 550. Continuing with this example, an operator can point the tip portion 550a of the elongate flexible device 550 at the target 552 (e.g., such that the target 552 is within or centered in a field of view of an image sensor of the elongate flexible device 550), and anatomic substructures can be identified based on the orientation or pose of the tip portion 550a. In FIG. 5, for example, the calyx 512b and/or the calyx 512c can be identified at step 130 of the method 100 (FIG. 1) as anatomic substructures that provide access to the target 552 based at least in part on the fact that the tip portion 550a of the elongate flexible device 550 is generally pointing at the calyces 512b and 512c while the tip portion 550a is directed toward the target 552.

In some embodiments, the system can identify one or more anatomic substructures automatically and/or based at least in part on input received from the operator. In these and other embodiments, the system can identify anatomic substructures based on one or more factors. For example, the system can identify (e.g., using the 3D model generated at step 110) anatomic substructures based on distance (e.g., shortest distance) between the target 552 and a distal opening of a calyx; the shape of access to the target 552 from a distal opening of a calyx (e.g., a direct or linear path may be appropriate for procedures using rigid instruments, a curved path may be appropriate for procedures using flexible instruments); and/or locations of sensitive tissue structures or other patient anatomy surrounding the anatomic structure. The system can identify anatomic substructures based on other factors, such as the position of the patient (e.g., identified using input received from a user via a user interface of the system). For example, for a PCNL procedure, a patient is typically laying on their back. Thus, the system can identify calyces (e.g., the calyces 512a-512c) that provide access to the target 552 via a posterior of the kidney (e.g., as opposed to calyces, such as the calyx 512d, the provides access to the target 552 via an anterior of the kidney).

In embodiments in which multiple targets are identified within the anatomic structure, the system can identify one or more anatomic substructures that provide access to several (e.g., all or a subset) of the targets. In other words, the system can identify anatomic substructures that provide access to the target(s) that would reduce or minimize the number of punctures required to reach all of the target(s). In embodiments in which a target is movable, the system can recommend moving the target to another location within the anatomic structure. This can be particularly helpful in embodiments in which no anatomic substructure provides suitable access to a target or in which another anatomic substructure would provide better access to a target. In such embodiments, the system can recommend moving a target to another location and can identify anatomic substructures that would provide suitable access to the other location. The recommended movement of the target can be presented to a user within a user interface as graphical guidance (e.g., arrows or other visual indicators) that visually depict a suggested movement of the target within the 3D model. The graphical guidance can be overlaid onto the 3D model within the user interface.

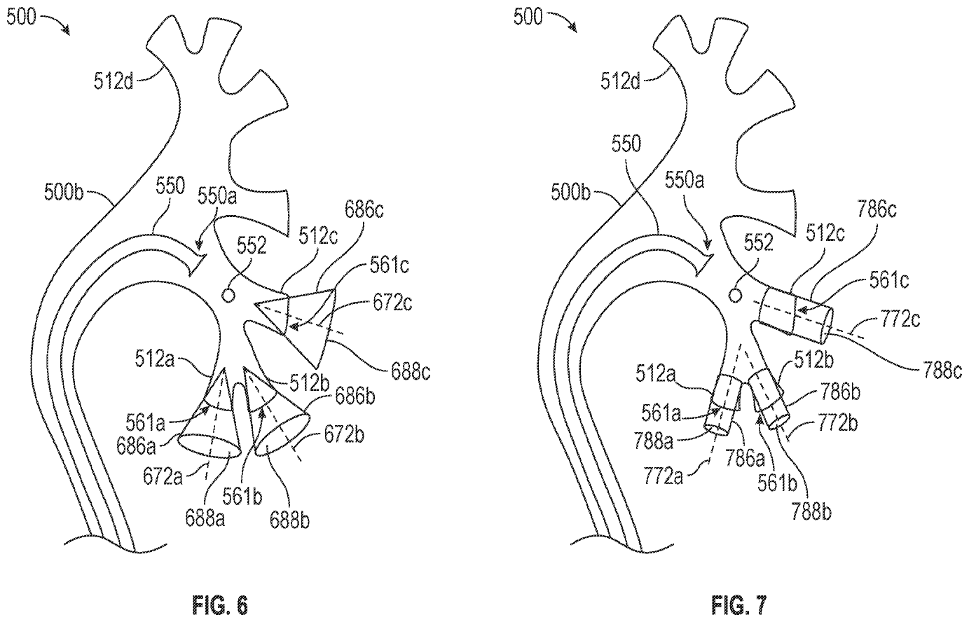

At step 140, the method 100 continues with identifying one or more approach paths to the target based at least in part on the anatomic substructures identified at step 130. An approach path can be a planned route for an access tool (e.g., a needle) to create an access path along which a medical instrument can be introduced to the target within the anatomic structure via minimally invasive techniques. For example, an approach path can provide a percutaneous route from a location external to a patient's body to a target or another location within an anatomic structure via an anatomic substructure identified at step 130. Step 140 of the method 100 is described in detail below with repeated reference to FIGS. 6-10, which illustrate various approach paths to the target 552 of FIG. 5 via anatomic substructures 512 in the 3D model, in accordance with various embodiments of the present technology.

In some embodiments, one or more approach paths can be based at least in part on the 3D anatomic model. For example, the system can determine, based at least in part on point cloud data used to generate the 3D anatomic model, a centerline of a calyx identified at step 130. The centerline can point directly out (e.g., the center of) a distal opening of the calyx and/or can extend from a point at or within the anatomic structure to a rendering of the patient's skin (or beyond). The centerline can indicate an optimal approach path along which an access tool can traverse to create an access path for a medical instrument. The optimal approach path can avoid sensitive tissue structures and/or other patient anatomy (e.g., ribs) or regions not suitable for inserting an access tool into the anatomic structure.

Referring to FIG. 6, for example, the calyces 512a-512c were identified at step 130 as anatomic substructures that provide access to the target 552. Thus, at step 140, the method 100 can include determining centerlines 672a-672c of the calyces 512a-512c, respectively. The centerlines 672a-672c extend along respective ones of the calyces 512a-512c and through (e.g., an estimate of the center of) the distal openings 561a-561c, respectively, of the calyces 512a-512c. In some embodiments, the centerlines 672a-672c can track projections of the calyces 512a-512c in the point cloud data and/or in other internal or external imaging of the calyces 512a-512c. As described in greater detail below, one or more of the centerlines 672a-672c can be displayed in (e.g., overlayed on) the 3D anatomic model and/or can provide an operator guidance when percutaneously inserting an access tool to the target 552.

In these and other embodiments, the system can generate a range of suitable approach paths for an access tool. In such embodiments, the system can generate a cone or another suitable shape that represents a set of reasonable angles or vectors that an access tool can enter the anatomic structure via an anatomic substructure. The cones can avoid sensitive tissue structures and/or other patient anatomy (e.g., ribs) or regions not suitable for inserting an access tool into the anatomic structure. For example, referring again to FIG. 6, the system can generate one or more cones 686a-686c. Each of the cones 686a-686c can represent a set of reasonable angles or vectors that an access tool can enter a respective one of the calyces 512a-512c via a respective one of the distal openings 561a-561c. More specifically, two-dimensional cross sections or faces 688a-688c of the cones 686a-686c at locations within the 3D anatomic model can represent a range of acceptable locations through which an access tool may pass when creating an access path to the target 552. In the illustrated embodiment, the faces 688a-688c gradually decrease in diameter as the approach paths draw nearer to the target 552 until, for example, the range of acceptable locations converges on the respective centerlines 672a-672c of the calyces 512a-512c.

In some embodiments, the cones 686a-686c can be based at least in part on the centerlines 672a-672c of the calyces 512a-512c, projections of the walls of the calyces 512a-512c, and/or on estimates of the diameters of the calyces 512a-512c. For example, a diameter of a two-dimensional cross section of the cone 686a can be limited by an estimated diameter of a two-dimensional cross section of the calyx 512a at a corresponding location within the 3D anatomic model. In some embodiments, the cones 686a-686c can extend from their respective points (e.g., at or within the anatomic structure) to any distance away from the points, including to any depth within the patient, to a rendering of a patient's skin, and/or to any point beyond the rendering of the patient's skin. Extending the cones 686a-686c distally toward a rendering or location of the patient's skin in the 3D model can be helpful, for example, to identify or recommend an appropriate puncture location and/or to ensure an optimal orientation and/or pose of an access tool before inserting the access tool into the patient.

As described in greater detail below, one or more of the cones 686a-686c can be displayed in (e.g., overlayed on) the 3D anatomic model and/or can provide an operator guidance when percutaneously inserting an access tool to the target 552. For example, the system can recommend one of more of the cones 686a-686c as an optimal approach path and/or as guidance for percutaneously inserting the access tool to the target 552 by displaying the cones 686a-686c to the operator. In embodiments in which the system displays or recommends more than one of the cones 686a-686c to the operator, the operator can select one of the displayed cones 686a-686c as a desired approach path for the access tool. In these and other embodiments, the operator can adjust a size, orientation, and another feature of any of the cones 686a-686c via user inputs on a user interface presented to the operator.

In some embodiments, one or more approach paths identified at step 140 can be based at least in part on simplified models of corresponding anatomic substructures identified at step 130. For example, referring to FIG. 7, the calyces 512a-512c identified at step 130 can be modeled as cylinders 786a-786c. Each of the cylinders 786a-786c can represent a range of insertion points, angles, or vectors that provide reasonable access into a respective one of the calyces 512a-512c via the distal openings 561a-561c. A diameter of each cylinder 786a-786c can be based at least in part on an estimate of the diameter of the respective one of the calyces 512a-512c (e.g., using point cloud data and/or internal or external imaging of the respective one of the calyces 512a-512c). Additionally, or alternatively, a diameter of each cylinder 786a-786c can be based at least in part on an estimate of a projection of the walls of the respective one of the calyces 512a-512c (e.g., when the respective one of the calyces 512a-512c cannot be surveyed or navigated by the elongated flexible device due to, for example, blockage of the respective one of the calyces 512a-512c by the target 552). Similar to the cones described above, two-dimensional cross sections or faces 788a-788c of the cylinders 786a-786c at locations within the 3D anatomic model can represent a range of acceptable locations through which an access tool may pass when creating an access path to the target 552. In some embodiments, the cylinders 786a-786c can extend from the anatomic structure to any distance away from the anatomic structure, including to any depth within the patient, to a rendering of a patient's skin, and/or to any point beyond the rendering of the patient's skin. The cylinders can avoid sensitive tissue structures and/or other patient anatomy (e.g., ribs) or regions not suitable for inserting an access tool into the anatomic structure. Extending the cylinders 786a-786c toward a rendering or location of the patient's skin in the 3D model can be helpful, for example, to identify or recommend an appropriate puncture location on the patient and/or to ensure an optimal orientation and/or pose of an access tool before inserting the access tool into the patient.

In some embodiments, the cylinders 786a-786c can be generated based at least in part on the centerlines 672a-672c (FIG. 6) of the calyces 512a-512c. Additionally, or alternatively, one or more optimal approach paths or centerlines 772a-772c (FIG. 7) can be determined after generating the cylinders 786a-786c. For example, the centerlines 772a-772c can be based at least in part on the cylinders 786a-786c. More specifically, the system can determine the centerlines 772a-772c of each of the cylinders 786a-786c based on characteristics (e.g., diameter, pose, etc.) of the cylinders 786a-786c.

Additionally, or alternatively, one or more of the cylinders 786a-786c can be displayed in (e.g., overlayed on) the 3D anatomic model and/or can provide an operator guidance when percutaneously inserting an access tool to the target 552. In such embodiments, a user can (a) identify a center of calyx and/or a corresponding cylinder model to facilitate the system generating a centerline of the calyx or the cylinder model; (b) adjust the diameter, orientation, and/or other features of a cylinder model via user inputs on a user interface; and/or (c) adjust the location, orientation, and/or other features of a centerline or optimal approach path via user inputs on the user interface. In some embodiments, the system can recommend one of more of the cylinders 786a-786c as an optimal approach path and/or as guidance for percutaneously inserting the access tool to the target 552 by displaying the cylinders 786a-786c and/or the respective centerlines 772a-772c to the operator. In embodiments in which the system displays or recommends more than one of the cylinders 786a-786c and/or more than one of the centerlines 772a-772c to the operator, the operator can select one of the displayed cylinders 786a-786c and/or one of the displayed centerlines 772a-772c as a desired approach path for the access tool.

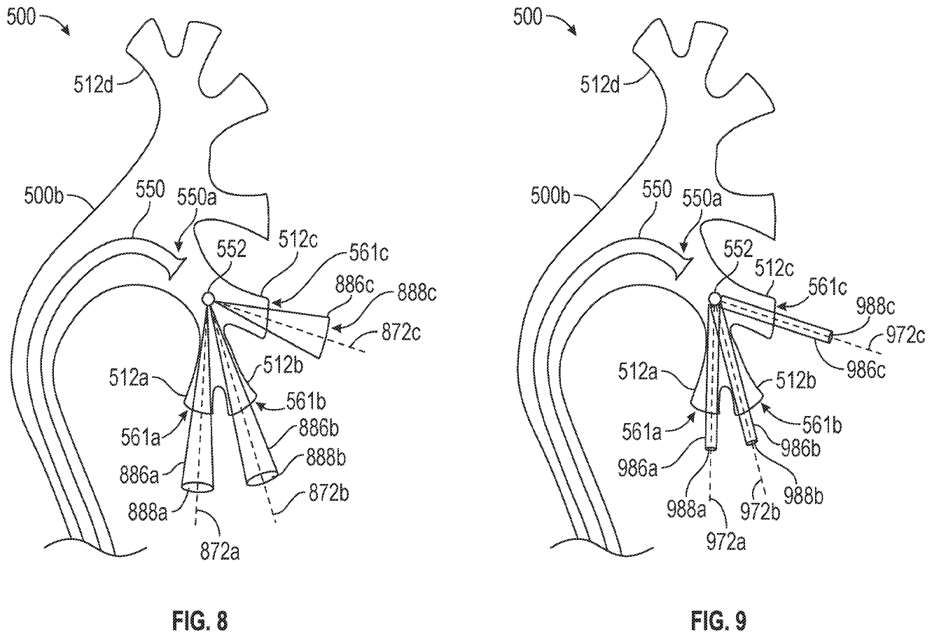

Apart from using the location of the target 552 to initially identify one or more anatomic substructures that provide access to the target 552, in this embodiment, the system does not use the location of the target 552 to generate the centerlines, cones, and cylinders described above. Rather, the system merely uses the 3D model (or the underlying point cloud data, imaging, and/or other data) of the anatomic substructures to identify and generate ranges of optimal approach paths for an access tool to enter the anatomic structure via the anatomic substructures. In other embodiments, the system can use the location of the target 552 to generate centerlines, cones, and/or cylinders representing ranges of optimal approach paths that converge on the target 552. For example, one or more approach paths can be identified based at least in part on the location of a target and characteristics of an anatomic substructure identified at step 130. As a specific example, the system can generate an optimal approach path (e.g., a centerline) by determining a path that extends from a center or another portion of the target 552 to an exterior of the anatomic structure via a center or another portion of (e.g., a distal opening of) an anatomic substructure. This is shown in FIGS. 8 and 9 in which centerlines 872a-872c (FIG. 8) and centerlines 972a-972c (FIG. 9) extend from a center of the target 552 to locations external the kidney via centers of the distal openings 561a-561c of the calyces 512a-512c. In some embodiments, the point cloud data and/or a projection of the walls of the calyces 512a-512c can be used to determine a location, orientation, diameter, and/or other features of the distal openings 561a-561c of the calyces 512a-512c.

Additionally, or alternatively, the system can generate cones 886a-886c (FIG. 8) and/or cylinders 986a-986c (FIG. 9) based at least in part on the centerlines 872a-872c and 972a-972c, respectively. For example, the centerlines 872a-872c can serve as centerlines of the cones 886a-886c, and the centerlines 972a-972c can serves as centerlines of the cylinders 986a-986c. Each of the cones 886a-886c and the cylinders 986a-986c can represent a set of reasonable angles or vectors that an access tool can approach the target 552. More specifically, two-dimensional cross sections or faces 888a-888c (FIG. 8) of the cones 886a-886c and/or two-dimensional cross sections or faces 988a-988c (FIG. 9) of the cylinders 986a-986c at locations within the 3D anatomic model can represent a range of acceptable locations through which an access tool may pass when creating an access path to the target 552 through a distal opening of a calyx. Unlike the points of the cones 686a-686c (FIG. 6) and the proximal end faces of the cylinders 786a-786c (FIG. 7) that are not necessarily positioned at the target 552, the point of the cones 886a-886c of FIG. 8 and the end faces of the cylinders 986a-986c can be positioned at the location of the target 552. Thus, as an access tool navigates the ranges of acceptable approach paths defined by one of the cones 886a-886c and/or by one of the cylinders 986a-986c, the access tool creates an access path that will enter one of the calyces 512a-512c via a respective one of the distal openings 561a-561c and that will converge upon and/or terminate at the location of the target 552. In this regard, the proximal end faces (e.g., the face closest to the target 552) of the cylinders 986a-986c can be positioned and/or sized such that any acceptable approach path that intersects the proximal end faces would position an access tool close enough to the target 552 to provide a medical instrument access to the target 552.

In some embodiments, the optimal approach paths included in each of the cones 886a-886c and each of the cylinders 986a-986c can avoid sensitive tissue structures and/or other patient anatomy (e.g., ribs) or regions not suitable for inserting an access tool into the anatomic structure. In these and other embodiments, the cones 886a-886c and/or the cylinders 986a-986c can be constrained (a) by the walls of the respective calyces 512a-512c and/or (b) by the cones 686a-686c (FIG. 6) or the cylinders 786a-786c (FIG. 7), respectively. For example, referring to FIG. 8, the diameter, orientation, pose, and/or other features of the cones 886b can be constrained such that (a) the point of the cone 886b is positioned at the location of the target 552; (b) the cone 886b does not intersect with walls of the calyx 512b; (c) the diameters of portions of the cone 886b internal the anatomic structure is limited by the diameters of corresponding portions of the calyx 512b and/or the diameter of the distal opening 561b of the calyx 512b; and/or (d) a portion of the cone 886b external the anatomic structure falls within a portion of the cone 686b (FIG. 6) external the anatomic structure. Thus, in these embodiments, the cone 886b can represent a range of optimal approach paths that (a) enter the calyx 512b via the distal opening 561b and (b) provide a more direct or linear path to the target 552 than approach paths included in the cone 686b.

Similarly, referring to FIG. 8, the diameter, orientation, pose, and/or other features of the cylinder 986b can be constrained such that (a) the proximal end face of the cylinder 986b is positioned at the location of the target 552; (b) the cylinder 986b does not intersect with walls of the calyx 512b; (c) the diameter of a portion of the cylinder 986b internal the anatomic structure is limited by the diameters of corresponding portions of the calyx 512b and/or the diameter of the distal opening 561b of the calyx 512b; and/or (d) a portion of the cylinder 986b external the anatomic structure falls within a portion of the cylinder 786b (FIG. 7) external the anatomic structure. Thus, in these embodiments, the cylinder 986b can represent a range of optimal approach paths that (a) enter the calyx 512b via the distal opening 561b and (b) provide a more direct or linear path to the target 552 than approach paths included in the cylinder 786b.

In other embodiments, a centerline, a cone, and/or a cylinder can be based at least in part on a location of a feature of the anatomic structure (e.g., the location of an end of the renal pelvis of a kidney), an end of the respective anatomic substructure (e.g., a proximal or distal end or opening of a respective calyx, and/or another location within or feature of the anatomic structure. For example, a point of a cone or the proximal end face of a cylinder can be positioned at the location of the end of the renal pelvis, at the location of the distal opening of the respective calyx, or at another location (e.g., within the anatomic structure).

In these and still other embodiments, one or more approach paths can be based at least in part on the elongate flexible device positioned within the anatomic structure in addition to or in lieu of the 3D anatomic model. For example, after locating a target using the flexible elongate device (as described above at step 120 of the method 100), the elongate flexible device can be used to generate and/or provide an approach path for guidance of an access tool to the target. In particular, the elongate flexible device can be used to locate a calyx proximate to the target. When such a calyx is identified, a tip portion of the elongate flexible device can be pointed at the distal end of the calyx to determine a location of the distal end of the calyx. In turn, the system can generate an approach path that extends from the elongate flexible device, along the chosen calyx, and out the distal end of the calyx.

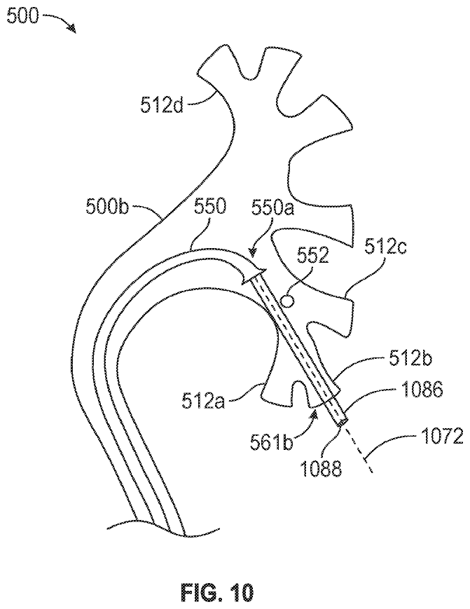

Referring to FIG. 10 for the sake of clarity, an elongate flexible device 550 carrying an endoscopic camera can be used to visually identify the target 552 within the anatomic structure. The elongate flexible device 550 can then be used to visually identify a calyx (e.g., the calyx 512b) proximate the target. After identifying the calyx, the tip portion 550a of the elongate flexible device 550 can directed toward a distal opening of the calyx (e.g., the distal opening 561b of the calyx 512b) and/or along a centerline of the identified calyx. The system can then use a vector provided by a shape sensor or another sensor of the elongate flexible device to determine an approach path (e.g., the approach path 1072) and/or a centerline of the calyx. The generated line can serve as an approach path along which an access tool percutaneously inserted into the patient can travel to reach the target 552. As shown in FIG. 10, the approach path 1072 extends from the elongate flexible device 550 within the anatomic structure to an exterior of the anatomic structure via the distal opening 561b of the calyx 512b. In some embodiments, the system or an operator can attempt to center the target 552 in a field of view of an image sensor positioned at the tip portion 550a of the elongate flexible device 550 such that the approach path 1072 intersects the target 552 between the elongate flexible device 550 and the distal opening 561b of the calyx 512b. In such embodiments, an access tool following the approach path 1072 can intersect the target 552 before reaching the elongate flexible device 550.

In some embodiments, the approach path 1072 can be used to generate a cone or cylinder similar to the cones and cylinders described above. For example, the approach path 1072 can be used to generate a cylinder 1086 representing a range of acceptable approach paths that provide reasonable access into the calyx 512b via the distal opening 561b and/or to the target 552. A diameter of the cylinder 1086 can be based at least in part on an estimate of the diameter of the calyx 512b, a diameter of the elongate flexible device, and/or other factors (e.g., acceptable puncture locations and/or locations of sensitive tissue structures external the anatomic structure). Additionally, or alternatively, a diameter of the cylinder 1086 can be based at least in part on an estimate of a projection of the walls of the calyx 512b. Two-dimensional cross sections or faces 1088 of the cylinder 1086 at locations within the 3D anatomic model can represent a range of acceptable locations through which an access tool may pass when creating an access path to the elongate flexible device 550 and/or to the target 552.

Any of the above approach paths can be determined and/or recommended to an operator based at least in part on various factors, such as path length (e.g., the shortest path to the target), path shape (e.g., a straight path may be appropriate for procedures using rigid instruments, a curved path may be appropriate for procedures using flexible instruments), size of anatomic substructure (e.g., a calyces having a larger diameters may provide greater or easier access to a target than calyces having smaller diameters), avoiding intersecting with or passing too close to sensitive tissue structures, avoiding entering or intersecting regions marked off (e.g., by a physician) as not suitable for a percutaneous puncture or access path, and/or optimal approach to a target organ. In these and other embodiments, the factors can include number of punctures. For example, in embodiments in which there are multiple targets (e.g., multiple kidney stones), the system can identify an approach path or group of approach paths that provide access to each of the targets and that reduce or minimize the number of percutaneous punctures required to reach all of the targets. In some embodiments, step 140 further includes determining an insertion position and/or angle for an access tool (e.g., a needle, cannula, etc.) to create an initial puncture, incision, or other opening for the access path. The insertion position and/or angle can be aligned with (e.g., parallel to) the trajectory of the approach path. The system can display all or a subset of the reasonable approach paths and/or access tool insertion positions/angles that the system identifies to an operator on a user interface, and/or the system can highlight which of the reasonable approach paths and/or access tool insertion positions/angles are most optimal based on one or more of the factors discussed above.

Optionally, as discussed above, step 140 of the method 100 (FIG. 1) can include displaying the determined approach path(s) to an operator so the operator can review the approach path(s) and provide feedback, if appropriate. For example, step 140 can include presenting a graphical user interface including the approach path(s), cones, and/or cylindrical models overlaid onto the 3D anatomic model. The operator can view the approach paths and provide feedback to accept, reject, or modify an approach path (e.g., via an input device such as a mouse, keyboard, joystick, touchscreen, etc.). In some embodiments, step 140 includes generating or recommending multiple approach paths (e.g., multiple entry points/paths, different path lengths, shapes, insertion locations, etc.), and the operator can select a particular approach path to be used in the procedure based on desirability (e.g., distance to critical structures, path length, etc.).

With reference again to FIG. 1, at step 150, the method 100 optionally includes updating the 3D anatomic model and/or approach path, based on intraoperative data (e.g., image data, location data, user input, etc.). Updates to the model may be appropriate, for example, if the target, anatomic structure, and/or sensitive tissue structures move or otherwise change during the procedure. Additionally, the 3D anatomic model can be updated to more accurately conform to the actual geometry and/or locations of the target, anatomic structure, and/or sensitive tissue structures. For example, as previously discussed, the geometry and/or locations of the sensitive tissue structures in the 3D anatomic model can be initial estimates that are subsequently updated once intraoperative data is available. As another example, when the target moves within anatomy, the target location in the 3D anatomic model can be updated, e.g., by moving a distal section of the elongate flexible device to a plurality of different positions to maintain the target within the field of view of a camera coupled to the elongate flexible device. The elongate flexible device (and the camera coupled thereto) may be user controlled (e.g., manually navigated and/or robotically controlled via operator control through an input device) and/or automatically controlled (e.g., using a pre-programmed set of instructions from a robotic system). The approach path can also be updated to account for the changes to the 3D anatomic model, if appropriate. The 3D anatomic model and/or approach path can be updated at any suitable frequency, such as continuously, periodically at predetermined time intervals (e.g., once every x number of seconds, minutes, etc.), when new sensor data is received, when significant changes are detected (e.g., if the target moves), in response to user input, and/or combinations thereof. As discussed in greater detail below with respect to step 170, the 3D model and/or guidance displayed on a user interface presented to a user can additionally or alternatively be updated based, for example, on user input received via the user interface and/or on a change in the position, orientation, and/or pose of an access tool.

In some embodiments, the 3D anatomic model is updated based on intraoperative image data obtained during the medical procedure, such as CT data, fluoroscopy data, ultrasound data, etc. The image data can be obtained by an external imaging system, by an imaging device within the patient's body (e.g., carried by the elongate flexible device or by an access tool navigating an approach path), or a combination thereof. The image data can be analyzed to identify the current geometry and/or locations of the target, anatomic structure, and/or sensitive tissue structures, such as based on user input, using computer vision and/or machine learning techniques, and/or a combination thereof. The current geometry and/or locations of the target, anatomic structure, and/or sensitive tissue structures can be compared to the 3D anatomic model to identify any significant differences (e.g., changes in shape, size, location, etc.). If appropriate, the 3D anatomic model can be revised to reflect the current geometry and/or locations depicted in the image data. Optionally, the revisions can be presented to the operator for feedback (e.g., approval, rejection, or modification) before being incorporated in the model.

Optionally, step 150 can include registering the intraoperative data to the 3D anatomic model so that the geometry and/or locations in the intraoperative data can be mapped onto the model. For example, in embodiments where the intraoperative data includes image data obtained with external imaging systems, the registration process can include obtaining image data of the elongate flexible device or a portion thereof (e.g., the distal end portion) and identifying the elongate flexible device in the image data. The identification can be performed automatically (e.g., using computer vision and/or machine learning techniques), based on user input, or combinations thereof. Optionally, the elongate flexible device can be positioned in a shape to facilitate identification (e.g., a hooked shape). Examples of registration processes based on image data of an elongate flexible device are provided in International Publication No. WO 2017/139621, filed Feb. 10, 2017, disclosing “Systems and Methods for Using Registered Fluoroscopic Images in Image-Guided Surgery,” which is incorporated by reference herein in its entirety. In some embodiments, the registration process of step 150 can alternatively or additionally be performed at a different stage in the method 100, e.g., as part of any of steps 110-140.

At step 160, the method 100 optionally includes tracking a pose of an access tool relative to the 3D anatomic model. As previously discussed, the access tool can be a needle or other suitable medical instrument for creating an access path (e.g., by navigating along an approach path), and the tracked pose (e.g., position, orientation, location) can be used to guide an operator in deploying the access tool along an approach path, as discussed further below. The access tool may be positioned manually, the access tool may be robotically controlled by operator control through an input device, or the access tool may be robotically controlled automatically using a pre-programmed set of instructions from a robotic system (as will be described in further detail below with reference to FIGS. 13-14B).

The pose of the access tool can be tracked in many different ways, such as using a localization sensor (e.g., shape sensor, EM sensor), an imaging device (e.g., ultrasound, fluoroscopy, CT), a support structure having a known spatial and/or kinematic relationship with the access tool (e.g., a mechanical jig, needle guide, insertion stage, etc.), or suitable combinations thereof. For example, the access tool can include a localization sensor configured to generate location data of the access tool. The localization sensor can be configured to be removably coupled to the access tool (e.g., a sensor fiber or other component inserted within a working channel or lumen) or can be permanently affixed to the access tool. Additional examples of techniques for incorporating a localization sensor in an access tool are provided in U.S. Pat. No. 9,636,040, filed Jan. 28, 2013, disclosing “Steerable Flexible Needle with Embedded Shape Sensing,” which is incorporated by reference in its entirety.

In some embodiments, the access tool localization sensor is registered to the flexible device localization so that the pose of the access tool can be tracked relative to the elongate flexible device (and thus, the reference frame of the 3D anatomic model). The registration can be performed in various ways. For example, the first and second localization sensors can be placed in a known spatial relationship with each other during a setup procedure, e.g., manually by the operator and/or using a 3D guide, block, plate, etc., that includes cutouts or other patterning for positioning the sensors in a predetermined configuration. As another example, the first and second localization sensors can be touched to the same set of reference points on the patient's body and/or another object. In a further example, the first and second localization sensors can be coupled to the same support structure such that their relative spatial configuration is known. For instance, the proximal end portions of both sensors can be mounted to the same insertion stage or other structural support. In still another example, the first and second localization sensor can be coupled to different support structures, but the spatial configuration and/or kinematics between the different structures is known and can be used to calculate the spatial relationship between the sensors. For instance, the proximal end portion of the first localization sensor can be mounted to a first insertion stage, robotic arm, etc., while the proximal end portion of the second localization sensor can be mounted to a second insertion stage, robotic arm, etc. As yet another example, the first and second localization sensors can be or include a receiver-transmitter pair, and the signals communicated between the receiver-transmitter pair can be used to determine the spatial relationship between the sensors.

In other embodiments, however, the localization sensor used to track the access tool can be the same localization sensor used to generate the survey location data of the elongate flexible device in step 110. In such embodiments, the localization sensor can be a removable sensor (e.g., a sensor fiber) configured to be sequentially coupled to (e.g., inserted in a working lumen of) the elongated flexible device and the access tool. The localization sensor can first be coupled to the elongate flexible device to obtain data of the anatomic structure and target, as previously discussed with respect to steps 110 and 120. In some embodiments, once the target is detected (e.g., based on user input, image data, etc., as described above), the elongate flexible device is oriented toward the target and the localization sensor is used to record the pose of the elongate flexible device. The recorded pose can be used to determine the location of the target with respect to the elongate flexible device and/or 3D anatomic model, as described above. Subsequently, the localization sensor can be withdrawn from the elongate flexible device and coupled to the access tool to track the pose of the access tool, in connection with step 160. In some embodiments, because the same localization sensor is used for both the elongate flexible device and the access tool, no registration is needed to map the access tool pose data to the 3D anatomic model.

As another example, the access tool can include an imaging device (e.g., an ultrasound device) configured to generate image data (e.g. 3D Doppler images). The imaging device can be removably coupled to the access tool (e.g., inserted within a working channel or lumen) or can be permanently affixed to the access tool. The image data can be used to generate a 3D representation of the patient anatomy in the reference frame of the access tool. Subsequently, the 3D representation can be registered or otherwise compared to the 3D anatomic model to determine the pose of the access tool relative to the 3D anatomic model and/or update the 3D anatomic model and virtual image of the access tool within the 3D anatomic model.

In a further example, the access tool can be tracked using intraoperative image data (e.g., fluoroscopy, CT) generated by an imaging device separate from the access tool (e.g., an external imaging system). Depending on the particular imaging modality used, the image data can include views of the access tool from multiple imaging planes to facilitate continuous tracking (e.g., for fluoroscopy, multiple 2D views may be needed to track the 3D pose of the access tool). The access tool can be automatically or semi-automatically tracked in the image data based on the known geometry of the access tool, fiducials or other markers on the access tool, user input, etc. Optionally, the access tool can include a localization sensor, and the survey location data generated by the localization sensor can be used as guidance for orienting the imaging device to capture images of the access tool (e.g., for fluoroscopy, the imaging device can be adjusted so the access tool is parallel to the fluoroscopic imaging plane, which may be more suitable for tracking purposes). The intraoperative image data can then be registered to the 3D anatomic model so the pose of the access tool in the image data can be determined relative to the 3D anatomic model (e.g., using the techniques previously described in step 140). Alternatively, or in combination, the imaging device can obtain image data of the access tool together with the elongate flexible device so the pose of the access tool can be determined relative to the elongate flexible device (which can be in the same reference frame as the 3D anatomic model).

At step 170, the method 100 can include providing guidance for deploying the access tool to create the access path. The guidance can be presented to the user as a user interface displaying various information, such as a representation of the 3D anatomic model including the anatomic structure, target, and/or nearby sensitive tissue structures. Additionally, the user interface can show the locations of various medical instruments with respect to the 3D anatomic model, such as including virtual renderings or representations representing the real time locations of the elongate flexible device and/or the access tool. The virtual rendering of the elongate flexible device can be based at least in part on shape data and/or can be displayed on or within the 3D anatomic model. Optionally, the user interface can display the 3D anatomic model from a plurality of different virtual views, such as a global view showing the entire anatomic region, an access tool point of view, and/or an elongate flexible device point of view.

The user interface can also show the approach path determined in step 140 (e.g., as a virtual line or similar visual element overlaid onto the 3D anatomic model). The user interface can show other guidance (e.g., centerlines, cylinders, cones, navigation rings, etc.) in addition to or in lieu of the approach path. In some embodiments, the guidance can be overlaid onto the 3D anatomic model. In these and other embodiments, more than one potential approach path and/or corresponding guidance can be shown in the user interface. For example, if multiple suitable approach paths to a target exist, each of the approach paths and/or associated guidance (e.g., centerlines, cones, navigation rings from a rendering or location of the patient's skin in the 3D anatomic model to the target) can be simultaneously displayed. Continuing with this example, an optimal or recommended approach path and/or associated guidance can be indicated and/or otherwise highlighted to the operator within the user interface.

As the operator positions the access tool relative to the patient's body (e.g., manually or via a robotically-controlled system), the user interface can provide instructions, feedback, notifications, alerts, etc., to guide the operator in inserting the access tool into the patient's body along the planned approach path. For example, the user interface can display a target insertion location (e.g., by displaying crosshairs in the 3D anatomic model corresponding to a location of an external site on the patient's body) and/or a target insertion angle or orientation for the access tool to make the initial puncture for the access path. Optionally, an operator can markup the patient's skin (e.g., with lines from an ink pen that is coupled to a localization sensor or that is used in combination with another tool having a localization sensor) and identify intersections between (a) valid percutaneous entry points or areas indicated by the sharpie lines and (b) the centerline, cones, and/or cylinders of the potential approach paths recommended by the system. The user interface can also show the current location and/or angle of the access tool (e.g., based on the tracked pose of the access tool of step 150) relative to the target site, a point of initial puncture, the sensitive tissue structures, and/or the anatomic structure, and, if appropriate, provide feedback (e.g., visual, audible, haptic, etc.) guiding the operator to adjust current location and/or angle of the access tool toward the target location and/or angle, respectively.

The user interface can track the current pose of the access tool with respect to the planned approach path, target, and/or local anatomy as the operator inserts the access tool into the patient's body. In some embodiments, the user interface outputs alerts or other feedback (e.g., visual, audible, haptic, etc.) if the access tool deviates from the planned approach path, approaches sensitive tissue structures, or otherwise requires correction. The user interface can be updated (e.g., as previously discussed with respect to steps 140 and 150) to provide real-time monitoring and feedback until the access tool reaches the target.

In some embodiments, guidance displayed on the user interface can be periodically updated. For example, when an operator selects a desired approach path from a display of multiple suitable approach paths, the guidance (e.g., the approach paths, centerlines, cones, cylinders, navigation rings, etc.) associated with the non-selected approach paths can be removed or hidden from the user interface. As another example, as the access tool is inserted into the patient or is moved (e.g., to approach or arrive at the target), a position, orientation, pose and/or other features of the representation of the access tool within the 3D anatomic model can be updated accordingly. As still another example, in the event a location of the target changes (e.g., intentionally or otherwise), the representation of the target in the 3D anatomic model can accordingly be updated to reflect the new location of the target. Additionally, or alternatively, the user interface can be updated in response to other events, such as receipt of user input (e.g., via input options displayed on the user interface) and/or identification of sensitive tissue structures or anatomy within the approach path (e.g., using an ultrasound or other sensor attached to or included in the access tool). For example, after a system identifies an approach path providing access to a target, an operator can modify the approach path via input options on the user interface, and a display of the approach path and corresponding guidance can be updated in the user interface. As a specific example, the system can recommend puncturing a patient's skin at a first location for navigating an access tool along a recommended approach path. The operator can subsequently change the first location to a second location (e.g., based on user clinical knowledge and experience, to avoid sensitive anatomy, etc.) via user input options on the user interface. In turn, the system (a) can calculate a new vector from the second location to the centerline of the calyx, a distal opening of the calyx, and/or the target; (b) can update the recommended approach path to correspond to the new vector; and/or (c) can update a display of the guidance in the user interface to correspond to the updated approach path.

In these and other embodiments, guidance displayed within the user interface can include navigation rings or hoops. Navigation rings can be displayed, for example, in the global view and/or in the access tool point of view. In some embodiments, the navigation rings can be displayed as a series of rings or as a see-through cylinder or cone and can be provided to aid an operator in navigating the access tool along an approach path to a target. For example, the navigation rings can be a series of rings that increase in diameter moving away from the target. Continuing with this example, an operator can use the navigation rings to facilitate navigating an access tool to a target by passing a tip of the access tool through the navigation rings in order, much like how video game players fly through a series of hoops positioned in the sky in virtual flying games. A spacing between adjacent navigation rings displayed on the user interface can be intentionally selected to provide an operator a sense of insertion depth and/or distance of the access tool. Additionally, or alternatively, at least two navigation rings can be visible within the user interface while an operator is navigating an access tool to the target (e.g., to provide an operator a sense of where next to navigate the tip of the access tool and/or a sense of how best to orient or pose the access tool to ensure that the tip of the access tool passes through the next navigation ring of the sequence.

In embodiments including navigation rings and tracking of the access tool, the user interface can be periodically updated based on a position, orientation, and/or pose of the access tool. For example, when an orientation or pose of the access tool aligns with the navigation rings, the navigation rings can be displayed using a first color (e.g., green) or pattern. When an orientation or pose of the access tool does not align with the navigation rings, the navigation rings displayed within the user interface can be updated to display the navigation rings using a second color (e.g., red) or pattern. A virtual projection of the orientation of pose of the access tool can be shown in the user interface. For example, a virtual line projecting away from the tip of the access tool and aligned with a longitudinal axis of the access tool can be shown in the user interface to provide an operator a sense of orientation or pose of the access tool (e.g., to indicate the current trajectory of the access tool relative to other model components shown in the user interface). In these and other embodiments, as the tip of an access tool is advanced through a navigation ring or a portion of a cone/cylinder, the user interface can be updated to remove a display of the navigation ring or the portion of the cone/cylinder.

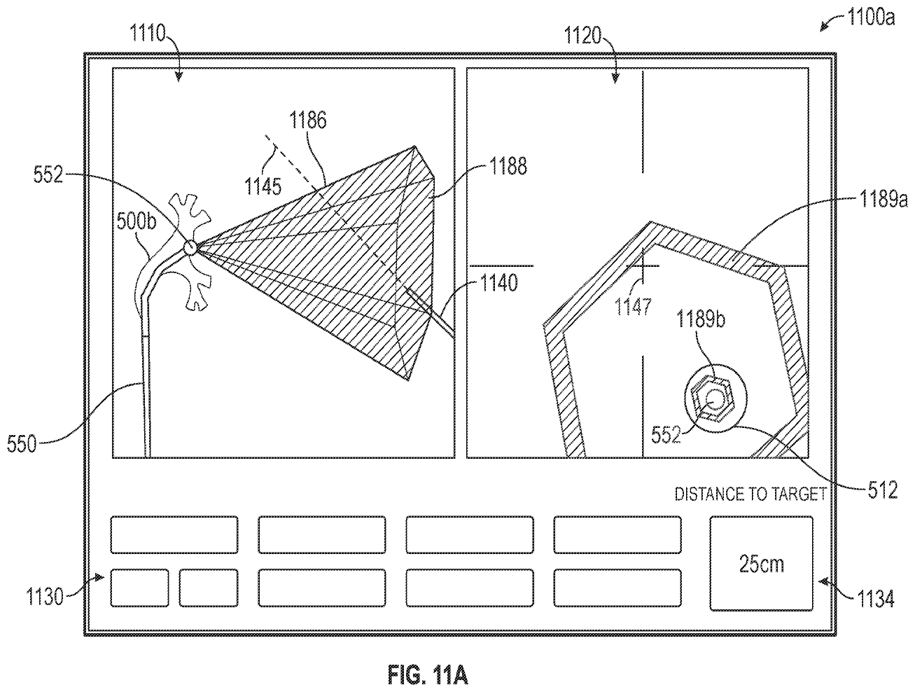

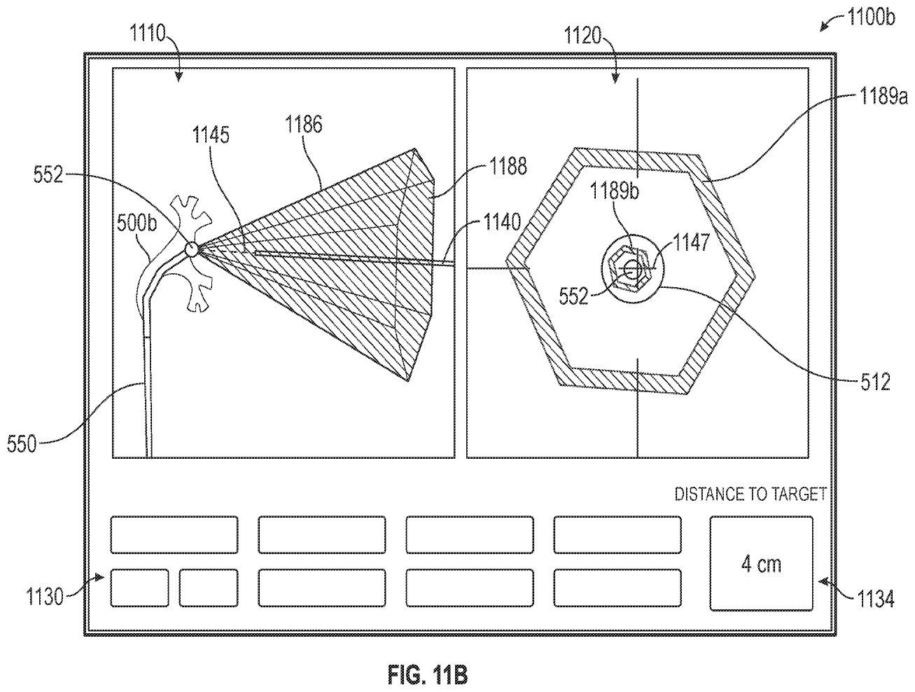

FIGS. 11A and 11B are partially schematic illustrations of various examples of user interfaces 1100a and 1100b, respectively, for providing guidance for deploying an access tool in accordance with embodiments of the present technology. The features of the interfaces 1100a and 1100b can be combined with each other and/or with any of the other embodiments described herein. Referring first to FIG. 11A, the user interface 1100a displays (a) a global view 1110; (b) an access tool point of view 1120; and (c) user input options 1130. In some embodiments, all or a portion of the user interfaces 1100a and 1100b may include a touchscreen which allows for user inputs received within the global view 1110 or access tool point of view 1120. The global view 1110 includes a display of anatomic substructures 500b (e.g., calyces, renal pelvis, ureter, etc.) of a 3D anatomic model of an anatomic structure (e.g., a kidney), a representation of an elongate flexible device 550 positioned within the anatomic structure, and a representation of an access tool 1140. The global view 1110 further includes a representation of a target (e.g., a kidney stone) within the anatomic structure and guidance in the form of a cone 1186 representing a set of appropriate approach paths for the access tool 1140 to traverse to arrive at or proximate the target 552 via a distal opening (not shown) of one of the calyces.

The access tool point of view 1120 illustrates a view from a tip or another position along the access tool 1140 of the global view 1110. For example, the access tool point of view 1120 can include crosshairs 1147 indicating a current location of the tip of the access tool 1140 with a view looking along a longitudinal axis of the access tool 1140. Multiple two-dimensional cross sections or faces 1188 of the cone 1186 from the global view 1110 are shown in the access tool point of view 1120 in the form of navigation rings 1189a and 1189b. Consistent with the discussion above, two-dimensional cross sections or faces 1188 of the cone 1186 at locations within the 3D anatomic model can represent a range of acceptable locations through which the access tool 1140 may pass when creating an access path to the target 552. Thus, the navigation rings 1189a and 1189b can be used to provide guidance to an operator while navigating the access tool 1140 to the target 552.

For example, in FIG. 11A, although the target 552 and a next navigation ring 1189b are visible in the access tool point of view 1120, the crosshairs 1147 is not aligned with the next navigation ring 1189b. This can easily be seen in the global view 1110 in which a projection or current trajectory (displayed as a dashed line 1145 in FIG. 11A) of the access tool 1140 diverges from an interior of the cone 1186. Therefore, although the operator may be able to pass the tip of the access tool 1140 through the closest navigation ring 1189a shown in the access tool point of view 1120, the operator will need to adjust the orientation and/or pose of the access tool 1140 to navigate the tip of the access tool 1140 through the next navigation ring 1189b shown in the access tool point of view 1120. For this reason, (a) the cone 1186, the access tool 1140, and/or the dashed line 1145 displayed in the global view 1110, and/or (b) the crosshairs 1147, the closest navigation ring 1189a, and/or the next navigation ring 1189b in the access tool point of view 1120 can be displayed in a second color (e.g., red) or with a second pattern. Additionally, or alternatively, the user interface 1100a can provide other feedback (e.g., visual, audio, haptic, etc.) to alert the operator that the access tool 1140 is currently off course.

Referring now to FIG. 11B, the user interface 1100b is similar to the user interface 1100a except that the access tool 1140 is aligned with an optimal approach path. In particular, the crosshairs 1147 in the access tool point of view 1120 are aligned with both the closest navigation ring 1189a and the next navigation ring 1189b. In addition, the dashed line 1145 in the global view 1110 representing a current orientation, pose, and/or trajectory of the access tool 1140 is within an interior of the cone 1186 and/or aligns with a centerline of the cone 1186. For this reason, (a) the cone 1186, the access tool 1140, and/or the dashed line 1145 displayed in the global view 1110, and/or (b) the crosshairs 1147, the closest navigation ring 1189a, and/or the next navigation ring 1189b in the access tool point of view 1120 can be displayed in a first color (e.g., green) or with a first pattern. Additionally, or alternatively, the user interface 1100b can provide other feedback (e.g., visual, audio, haptic, etc.) to indicate to the operator that the access tool 1140 is currently on course.

Referring to FIGS. 11A and 11B together, the user input options 1130 of the user interfaces 1100a and 1100b can include various software buttons or other elements that can receive input from the operator via touchscreen control. The user input options 1130 can additionally or alternatively display various information to the operator. For example, user input options 1130 can provide a distance 1134 (in real world units) between a tip of the access tool 1140 and the target 552 (e.g., along the approach path).

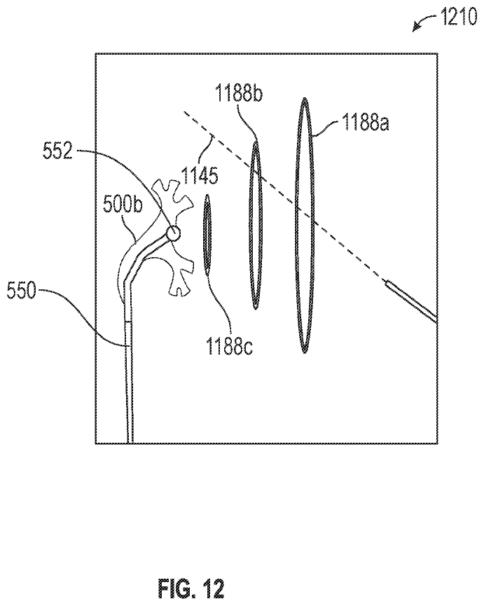

FIG. 12 is partially schematic illustration of another example global view 1210 for a user interface configured in accordance with embodiments of the present technology. For example, the global view 1210 can be included in the user interface 1100a of FIG. 11A in addition to or in lieu of the global view 1110. The global view 1210 is similar to the global view 1110 of FIG. 11A except that a series of navigation rings 1188a, 1188b, and 1188c are shown in lieu of the cone 1186. In some embodiments, the navigation rings 1188a-1188c can correspond to one or more of the navigations rings 1189a and/or 1189b shown in the access tool point of view 1120 in FIG. 11A. As shown in FIG. 12, the diameter of the rings 1188a-1188c decreases as the rings 1188a-1188c approach the target, consistent with the shape of the cone 1186 (FIG. 11A). Additionally, the rings 1188a-1188c are spaced apart from one another to provide the operator a sense of insertion depth and/or distance of the access tool 1140.

Referring again to step 170 of FIG. 1, in some embodiments, the graphical user interface displayed to the operator can include live image data from an imaging device, such as an external imaging system (e.g., fluoroscopy, ConeBeam, CT, etc.) and/or internal imaging device (e.g., endoscopic camera, ultrasound, etc.) within the patient's body. The imaging device can be the same imaging device used to update the 3D anatomic model (step 150) and/or track the access tool (step 160), or a different imaging device may be utilized. The image data can be presented together with the graphical representation of the 3D anatomic model so the operator can view and compare the actual pose of the access tool with the planned approach path.

In some embodiments, the graphical user interface also displays instructions, feedback, notifications, etc., for adjusting the imaging device to capture images of the access tool. This approach can be used in situations where different imaging planes are advantageous for different procedure steps. For example, when making the initial puncture with the access tool, the instructions can direct the operator to use an imaging plane normal or substantially normal to the planned approach path (e.g., an imaging plane that substantially aligns with the access tool point of view 1120 of FIGS. 11A and 11B while making the initial puncture) so that the approach path is shown as a point or small region on the patient's body. A normal imaging plane can help the operator place the distal tip of the access tool at the correct location. Optionally, a laser dot or similar visual indicator can be projected onto the patient's body to mark the insertion location.

Continuing with the above example regarding using an imaging plane normal or substantially normal to the planned approach path when making an initial puncture with an access tool, the instructions displayed on the graphical user interface can direct the operator (a) to position the access tool at a desired position, orientation, and/or pose for making the initial puncture and (b) to then rotate the imaging device until the access tool appears as a point within the imaging data. Additionally, or alternatively, the system can register the imaging data to the 3D anatomic model and then present instructions on the graphical user interface explaining to the operator how to adjust or move the imaging device to achieve an optimal imaging plane for viewing the approach path from the access tool point of view (e.g., when using fluoroscopy of CT, the user interface can indicate an optimal angle of rotation for the C-arm). In other embodiments, wherein the imaging device is controlled by the system, the system can automatically rotate/position the imaging device to achieve the optimal imaging plane. The optimal imaging plane can therefore be based at least in part on the planned approach path and/or on the 3D anatomic model. Further details regarding registering an access tool to a 3D anatomic model are provided in U.S. patent application Ser. No. 16/076,290, which is incorporated by reference herein in its entirety.

Once the initial puncture has been made, the instructions can then direct the operator to use an imaging plane parallel or substantially parallel to the planned approach path. A parallel imaging plane can provide a clearer view of the pose of the access tool as it is inserted into the body. In some embodiments, step 170 further includes monitoring the position and/or orientation of the imaging device (or a portion thereof, such as imaging arm) to instruct the operator on how to achieve the correct imaging plane and/or confirm that the correct imaging plane is being used.

At step 180, the method 100 continues with introducing a medical instrument to the target via the access path. In some embodiments, once the access tool has reached the target, the access tool is withdrawn so a medical instrument can be introduced to the target via the access path. Alternatively, the access tool can remain in the patient's body, and the medical instrument can be introduced into the patient's body via a working lumen or channel in the access tool. In other embodiments, however, the access tool itself can be used to treat the target, such that step 180 is optional and can be omitted.

The medical instrument can be any minimally invasive instrument or tool suitable for use in, for example, surgical, diagnostic, therapeutic, ablative, and/or biopsy procedures. For example, the medical instrument can be a suction tube, nephroscope, lithotripter, ablation probe, biopsy needle, or another suitable device used to treat the target. The positioning of the medical instrument may be performed manually, the medical instrument may be robotically controlled by operator control through an input device, or the medical instrument may be robotically controlled automatically using a pre-programmed set of instructions from a robotic system (as will be described further below with reference to FIGS. 13-14B).

Optionally, the graphical user interface provided in step 170 can also be used to guide the operator when introducing the medical instrument into the patient's body. For example, the pose of the medical instrument relative to the 3D anatomic model can be tracked. More specifically, the pose of the medical instrument relative to the 3D anatomic model can be tracked using the techniques described above in steps 160 and 170, such as a localization sensor coupled to medical instrument. Additionally, or alternatively, the pose of the medical instrument relative to the 3D anatomic model can be tracked by tracking (e.g., using sensors or encoders) the positions of manipulators or arms of a robotic system that is used introduce the medical instrument into the patient's body. Alternatively, or in combination, the graphical user interface can show live image data from a separate imaging device so the operator can visualize the location of the medical instrument within the patient anatomy. The image data can depict the medical instrument from a single imaging plane, or from multiple imaging planes. In some embodiments, for example, the medical instrument is imaged from an imaging plane parallel or substantially parallel to the access path, which may be helpful for visualizing the pose of the medical instrument. Optionally, the medical instrument itself can include an imaging device or other sensor system so the operator can monitor the location of the medical instrument and/or treatment progress from the point of view of the medical instrument.

Although the steps of the method 100 are discussed and illustrated in a particular order, the method 100 illustrated in FIG. 1 is not so limited. In other embodiments, the method 100 can be performed in a different order. In these and other embodiments, any of the steps of the method 100 can be performed before, during, and/or after any of the other steps of the method 100. For example, step 150 can be performed before, during, and/or after any of steps 160, 170, and/or 180; step 160 can be performed before, during, and/or after any of steps 110-150 or 170; and/or step 170 can be performed before, during, and/or after steps 150 and/or 160. Additionally, one or more steps of the method 100 can be repeated (e.g., any of steps 140-170).

Optionally, one or more steps of the method 100 illustrated in FIG. 1 can be omitted (e.g., steps 150 and/or 160). For example, in embodiments where the access tool is not tracked (e.g., step 160 is omitted), the method 100 can instead include registering the 3D anatomic model to live intraoperative image data (e.g., fluoroscopy data) so that the operator can track the location of the target, anatomic structure, and/or sensitive tissue structures relative to the live images. In such embodiments, the graphical user interface can overlay visual indicators (e.g., highlighting, shading, markings) representing the target, anatomic structure, and/or sensitive tissue structures onto the corresponding components in the live image data. The elongate flexible device and/or access tool can be visible in the live image data so that the operator can assess their locations relative to the patient anatomy. Thus, the location of the target can change, which will accordingly change the guidance of deploying an access tool to the location of the target. But guidance showing real-time alignment of the access tool to the guidance and/or the target may not be provided.

Moreover, a person of ordinary skill in the relevant art will recognize that the illustrated method 100 can be altered and still remain within these and other embodiments of the present technology. For example, although certain embodiments of the method 100 are described above with reference to a percutaneous access path, in other embodiments, the method 100 can be applied to other types of access paths. For example, the access tool can be introduced via an endoluminal access path, e.g., through a working channel or lumen of the elongate flexible device. In such embodiments, because the pose of the access tool corresponds to the pose of the elongate flexible device, the method 100 can omit determining an access path for the access tool (step 130) and/or tracking the pose of the access tool (step 150). Instead, the guidance provided in step 160 can focus on tracking and updating the location of the target, e.g., in case the target moves during the procedure.

Additionally, in other embodiments, the guidance provided by the method 100 can simply include directing the access tool toward the elongate flexible device (e.g., toward a distal end portion or other portion of the elongate flexible device near the target). In such embodiments, the method 100 does not need to determine a precise access path to the target (i.e., step 130 can be omitted). Instead, the method 100 can simply include tracking the relative locations of the access tool and elongate flexible device, such as by respective localization sensors on the access tool and elongate flexible device, a receiver on the access tool paired with a transmitted on the elongate flexible device (or vice-versa), and/or other suitable techniques. The guidance provided to the operator in step 160 can show the locations of the access tool and elongate flexible device relative to each other and/or to the 3D anatomic model. Optionally, the access tool can include an imaging device (e.g., an ultrasound device) and/or other sensor system to help the operator avoid sensitive tissue structures when inserting the access tool into the patient's body.

FIG. 13 is a simplified diagram of a teleoperated medical system 1300 (“medical system 1300”) configured in accordance with various embodiments of the present technology. The medical system 1300 can be used to perform any of the processes described herein in connection with FIGS. 1-12. For example, the medical system 1300 can be used to perform a medical procedure including mapping an anatomic structure with an elongate flexible device and creating an access path with an access tool, as previously discussed in connection with the method 100 of FIG. 1.

In some embodiments, the medical system 1300 may be suitable for use in, for example, surgical, diagnostic, therapeutic, or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

As shown in FIG. 13, the medical system 1300 generally includes a manipulator assembly 1302 for operating a medical instrument 1304 in performing various procedures on a patient P positioned on a table T. In some embodiments, the medical instrument 1304 may include, deliver, couple to, and/or control any of the flexible instruments described herein. The manipulator assembly 1302 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated.

The medical system 1300 further includes a master assembly 1306 having one or more control devices for controlling the manipulator assembly 1302. The manipulator assembly 1302 supports the medical instrument 1304 and may optionally include a plurality of actuators or motors that drive inputs on the medical instrument 1304 in response to commands from a control system 1312. The actuators may optionally include drive systems that when coupled to the medical instrument 1304 may advance the medical instrument 1304 into a naturally or surgically created anatomic orifice. Other drive systems may move the distal end of the medical instrument 1304 in multiple degrees of freedom, which may include three degrees of linear motion (e.g., linear motion along the X, Y, and Z Cartesian axes) and in three degrees of rotational motion (e.g., rotation about the X, Y, and Z Cartesian axes). Additionally, the actuators can be used to actuate an articulable end effector of the medical instrument 1304 for grasping tissue in the jaws of a biopsy device and/or the like. Actuator position sensors such as resolvers, encoders, potentiometers, and other mechanisms may provide sensor data to the medical system 1300 describing the rotation and orientation of the motor shafts. This position sensor data may be used to determine motion of the objects manipulated by the actuators.

The medical system 1300 also includes a display system 1310 for displaying an image or representation of the surgical site and the medical instrument 1304 generated by sub-systems of a sensor system 1308 and/or any auxiliary information related to a procedure including information related to ablation (e.g., temperature, impedance, energy delivery power levels, frequency, current, energy delivery duration, indicators of tissue ablation, etc.). The display system 1310 and the master assembly 1306 may be oriented so an operator O can control the medical instrument 1304 and the master assembly 1306 with the perception of telepresence.

In some embodiments, the medical instrument 1304 may include components of an imaging system, which may include an imaging scope assembly or imaging instrument that records a concurrent or real-time image of a surgical site and provides the image to the operator O through one or more displays of the medical system 1300, such as one or more displays of the display system 1310. The concurrent image may be, for example, a two or three-dimensional image captured by an imaging instrument positioned within the surgical site. In some embodiments, the imaging system includes endoscopic imaging instrument components that may be integrally or removably coupled to the medical instrument 1304. In some embodiments, however, a separate endoscope, attached to a separate manipulator assembly may be used with the medical instrument 1304 to image the surgical site. In some embodiments, the imaging system includes a channel (not shown) that may provide for a delivery of instruments, devices, catheters, and/or the flexible instruments described herein. The imaging system may be implemented as hardware, firmware, software, or a combination thereof which interact with or are otherwise executed by one or more computer processors, which may include the processors of the control system 1312.

The medical system 1300 may also include the control system 1312. The control system 1312 includes at least one memory and at least one computer processor (not shown) for effecting control the between medical instrument 1304, the master assembly 1306, the sensor system 1308, and the display system 1310. The control system 1312 also includes programmed instructions (e.g., a non-transitory machine-readable medium storing the instructions) to implement some or all of the methods described in accordance with aspects disclosed herein, including instructions for providing information to the display system 1310.

The control system 1312 may optionally further include a virtual visualization system to provide navigation assistance to the operator O when controlling the medical instrument 1304 during an image-guided surgical procedure. Virtual navigation using the virtual visualization system may be based upon reference to an acquired preoperative or intraoperative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 14A is a simplified diagram of a medical instrument system 1400 configured in accordance with various embodiments of the present technology. The medical instrument system 1400 includes an elongate flexible device 1402, such as a flexible catheter, coupled to a drive unit 1404. The elongate flexible device 1402 includes a flexible body 1416 having a proximal end 1417 and a distal end or tip portion 1418. The medical instrument system 1400 further includes a tracking system 1430 for determining the position, orientation, speed, velocity, pose, and/or shape of the distal end 1418 and/or of one or more segments 1424 along the flexible body 1416 using one or more sensors and/or imaging devices as described in further detail below.

The tracking system 1430 may optionally track the distal end 1418 and/or one or more of the segments 1424 using a shape sensor 1422. The shape sensor 1422 may optionally include an optical fiber aligned with the flexible body 1416 (e.g., provided within an interior channel (not shown) or mounted externally). The optical fiber of the shape sensor 1422 forms a fiber optic bend sensor for determining the shape of the flexible body 1416. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. No. 7,781,724, filed Sep. 26, 2006, disclosing “Fiber Optic Position and Shape Sensing Device and Method Relating Thereto”; U.S. Pat. No. 7,772,541, filed Mar. 12, 2008, disclosing “Fiber Optic Position and/or Shape Sensing Based on Rayleigh Scatter”; and U.S. Pat. No. 6,389,187, filed Apr. 21, 2000, disclosing “Optical Fiber Bend Sensor,” which are all incorporated by reference herein in their entireties. In some embodiments, the tracking system 1430 may optionally and/or additionally track the distal end 1418 using a position sensor system 1420. The position sensor system 1420 may be a component of an EM sensor system with the position sensor system 1420 including one or more conductive coils that may be subjected to an externally generated electromagnetic field. In some embodiments, the position sensor system 1420 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, filed Aug. 9, 1999, disclosing “Six-Degree of Freedom Tracking System Having a Passive Transponder on the Object Being Tracked,” which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g. fiber shape sensors, EM sensors, and/or the like) may be integrated within the medical instrument 1426 and used to track the position, orientation, speed, velocity, pose, and/or shape of a distal end or portion of medical instrument 1426 using the tracking system 1430.

The flexible body 1416 includes a channel 1421 sized and shaped to receive a medical instrument 1426. FIG. 14B, for example, is a simplified diagram of the flexible body 1416 with the medical instrument 1426 extended according to some embodiments. In some embodiments, the medical instrument 1426 may be used for procedures such as imaging, visualization, surgery, biopsy, ablation, illumination, irrigation, and/or suction. The medical instrument 1426 can be deployed through the channel 1421 of the flexible body 1416 and used at a target location within the anatomy. The medical instrument 1426 may include, for example, energy delivery instruments (e.g., an ablation probe), image capture probes, biopsy instruments, laser ablation fibers, and/or other surgical, diagnostic, or therapeutic tools. The medical instrument 1426 may be used with an imaging instrument (e.g., an image capture probe) within the flexible body 1416. The imaging instrument may include a cable coupled to the camera for transmitting the captured image data. In some embodiments, the imaging instrument may be a fiber-optic bundle, such as a fiberscope, that couples to an image processing system 1431. The imaging instrument may be single or multi-spectral, for example capturing image data in one or more of the visible, infrared, and/or ultraviolet spectrums. The medical instrument 1426 may be advanced from the opening of channel 1421 to perform the procedure and then be retracted back into the channel 1421 when the procedure is complete. The medical instrument 1426 may be removed from the proximal end 1417 of the flexible body 1416 or from another optional instrument port (not shown) along the flexible body 1416.

The flexible body 1416 may also house cables, linkages, or other steering controls (not shown) that extend between the drive unit 1404 and the distal end 1418 to controllably bend the distal end 1418 as shown, for example, by broken dashed line depictions 1419 of the distal end 1418. In some embodiments, at least four cables are used to provide independent “up-down” steering to control a pitch of the distal end 1418 and “left-right” steering to control a yaw of the distal end 1418. Steerable elongate flexible devices are described in detail in U.S. Pat. No. 9,452,276, filed Oct. 14, 2011, disclosing “Catheter with Removable Vision Probe,” and which is incorporated by reference herein in its entirety. In various embodiments, medical instrument 1426 may be coupled to drive unit 1404 or a separate second drive unit (not shown) and be controllably or robotically bendable using steering controls.

The information from the tracking system 1430 may be sent to a navigation system 1432 where it is combined with information from the image processing system 1431 and/or the preoperatively obtained models to provide the operator with real-time position information. In some embodiments, the real-time position information may be displayed on the display system 1310 of FIG. 13 for use in the control of the medical instrument system 1400. In some embodiments, the control system 1312 of FIG. 13 may utilize the position information as feedback for positioning the medical instrument system 1400. Various systems for using fiber optic sensors to register and display a surgical instrument with surgical images are provided in U.S. Pat. No. 8,900,131, filed May 13, 2011, disclosing “Medical System Providing Dynamic Registration of a Model of an Anatomic Structure for Image-Guided Surgery,” which is incorporated by reference herein in its entirety.

In some embodiments, the medical instrument system 1400 may be teleoperated within the medical system 1300 of FIG. 13. In some embodiments, the manipulator assembly 1302 of FIG. 13 may be replaced by direct operator control. In some embodiments, the direct operator control may include various handles and operator interfaces for hand-held operation of the instrument.

The systems and methods described herein can be provided in the form of tangible and non-transitory machine-readable medium or media (such as a hard disk drive, hardware memory, optical medium, semiconductor medium, magnetic medium, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. Programmed instructions may be implemented as a number of separate programs or subroutines, or they may be integrated into a number of other aspects of the systems described herein. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. In one embodiment, the control system supports wireless communication protocols such as Bluetooth, IrDA, HomeRF, IEEE 802.11, DECT, and Wireless Telemetry. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

Medical tools that may be delivered through the elongate flexible devices or catheters disclosed herein may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. Medical tools may include image capture probes that include a stereoscopic or monoscopic camera for capturing images (including video images). Medical tools may additionally house cables, linkages, or other actuation controls (not shown) that extend between their proximal and distal ends to controllably bend the distal ends of the tools. Steerable instruments are described in detail in U.S. Pat. No. 7,316,681, filed Oct. 4, 2005, disclosing “Articulated Surgical Instrument for Performing Minimally Invasive Surgery with Enhanced Dexterity and Sensitivity” and U.S. Pat. No. 9,259,274, filed Sep. 30, 2008, disclosing “Passive Preload and Capstan Drive for Surgical Instruments,” which are incorporated by reference herein in their entireties.

The systems described herein may be suited for navigation and treatment of anatomic tissues, via natural or surgically created connected passageways, in any of a variety of anatomic systems, including the lung, colon, stomach, intestines, kidneys and kidney calices, bladder, liver, gall bladder, pancreas, spleen, ureter, ovaries, uterus, brain, the circulatory system including the heart, vasculature, and/or the like.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art. The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

1. A method for providing guidance for percutaneous access to a target within an anatomic structure, the method comprising:
receiving point cloud data from a sensor system coupled to an internal instrument as the internal instrument is moved within the anatomic structure;
generating a 3D model including the anatomic structure, wherein the 3D model is based at least in part on the point cloud data;
receiving information for identifying a substructure within the 3D anatomic model, wherein the substructure provides access to the target;
determining an entry to the substructure;
determining an approach path through the entry; and
providing a graphical representation of the approach path.

2. The method of claim 1 wherein the approach path is based at least in part on geometry of the substructure.

3. The method of claim 1 wherein generating the 3D model further includes generating a representation of the target at a location within 3D model, and wherein the location of the target within the 3D model is based at least in part on localization data from the sensor system as the internal instrument is pointed toward the target.

4. The method of claim 1 wherein the information for identifying the substructure includes user input for selection of the substructure.

5. The method of claim 1 wherein the information for identifying the substructure includes position and orientation of the substructure relative to the target.

6. The method of claim 1 wherein the approach path is a linear, percutaneous approach path.

7. The method of claim 1 wherein the entry into the substructure is a distal opening of the substructure.

8. The method of claim 1, further comprising generating a cylindrical model of the substructure based at least in part on the point cloud data.

9. The method of claim 8 wherein the approach path is along a centerline of the cylindrical model.

10. The method of claim 9 wherein providing the graphical representation includes representing the approach path as a line along the centerline of the cylindrical model.

11. The method of claim 9 wherein providing the graphical representation includes representing the approach path as a cylindrical range of vectors around the centerline of the cylindrical model.

12. The method of claim 9 wherein providing the graphical representation includes representing the approach path as a cone of vectors converging at a point along the centerline of the cylindrical model.

13. The method of claim 12 wherein the point is at a proximal entry of the cylindrical model.

14. The method of claim 12 wherein the anatomic structure is a kidney and the point is near a renal pelvis of the kidney.

15. The method of claim 12 wherein a radius of the cone expands towards a distal opening of the cylindrical model.

16. The method of claim 12 wherein the radius of the cone is limited by a radius of the distal opening of the cylindrical model.

17. The method of claim 1 wherein the approach path is based at least in part on a location of the target.

18. The method of claim 17 wherein providing the graphical representation includes representing the approach path as a line from the target through a center of the distal opening of the substructure.

19. The method of claim 17 wherein providing the graphical representation includes representing the approach path as a cylindrical range of vectors from the target through the distal opening of the substructure.

20. The method of claim 17 wherein providing the graphical representation includes representing the approach path as a range of vectors at different angles converging at the target.

21-62. (canceled)
